(19) United States
(12) Patent Application Publication
(10) Pub. No.: US 2024/0325527 A1
(43) Pub. Date: Oct. 3, 2024

(54) METHODS FOR PROMOTING TISSUE REGENERATION

(71) Applicant: The Johns Hopkins University, Baltimore, MD (US)

(72) Inventors: Jennifer ELISSEEFF, Baltimore, MD (US); Jin Han, Baltimore, MD (US); Christopher Cherry, Baltimore, MD (US); Drew M. Pardoll, Baltimore, MD (US)

(21) Appl. No.: 18/292,353

(22) PCT Filed: Aug. 3, 2022
(86) PCT No.: PCT/US2022/039241

§ 371 Date: Jan. 26, 2024

Related U.S. Application Data
(60) Provisional application No. 63/322,030, filed on Mar. 21, 2022.
(60) Provisional application No. 63/230,386, filed on Aug. 6, 2021.

(51) Int. Cl. A61K 39/395; A61K 35/12; A61P 21/00

(52) U.S. Cl. CPC A61K 39/3955; A61K 35/12; A61P 21/00

(57) ABSTRACT

Recovering tissue repair capacity that is lost with aging represents a significant medical challenge. The present disclosure relates to methods for promoting wound healing, tissue repair, or tissue regeneration in a subject by administering a subject in need thereof, a pharmaceutically effective amount of at least one IL-17 antagonist and at least one regenerative therapy.

### RELATED APPLICATION INFORMATION

This application claims priority to U.S. Application No. 63/230,386 filed on Aug. 6, 2021 and U.S. Application No. 63/322,030 filed on Mar. 21, 2022, the contents of each of which are herein incorporated by reference.

### STATEMENT OF GOVERNMENT INTEREST

This invention was made with government support under grant 4134401-21-0075 awarded by the National Institutes of Health. The government has certain rights in the invention.

### FIELD

The present disclosure relates to methods for promoting wound healing, tissue repair, or tissue regeneration in a subject by administering a subject in need thereof, a pharmaceutically effective amount of at least one IL-17 antagonist and at least one regenerative therapy.

### BACKGROUND

Aging is associated with decreased tissue function and a compromised response to tissue damage that leads to longer recovery and frequently dysfunctional tissue repair regardless of tissue type¹⁻³. Reduced healing capacity with increasing age was recognized as early as 19324. The variability in time required for tissue repair and quality increases with age in both preclinical models and patients⁵⁻⁷, consistent with the variability in biological signatures of aging. Multi-omic analyses of cellular and molecular profiles of organisms over lifespan implicate changes in gene expression, metabolism, DNA methylation and other epigenetic factors in age-associated pathologies including impaired wound healing^{2,3}. Recovering tissue repair capacity that is lost with aging represents a significant medical challenge.

The composition and phenotype of cells responding to tissue damage changes with age. In skin wounds, the number of fibroblasts responding to injury is greater in older mice and the fibroblasts have reduced phenotypic heterogeneity compared to the wounds in younger counterparts⁸. In the case of muscle tissue, the number and activity of muscle stem cells decreases with age leading to sarcopenia and impaired muscle healing after injury⁹. However, the functionality of aged muscle stem cells can be restored ex vivo to recover healing capacity after re-injection in vivo, suggesting that endogenous repair capacity is retained but the aging tissue environment impedes repair⁹. Similarly, repair in the aging retina could be restored by targeting age-related epigenetic changes¹⁰, again suggesting that regeneration capacity remains with increasing age despite decreased cell numbers and inhibitory factors.

Regenerative medicine and tissue engineering approaches are designed to enhance repair and restore tissue function. While many patients needing regenerative medicine technologies are older, the influence of age-related physiological changes on regenerative medicine therapeutic responses remains unexplored. In fact, age-related changes may be, in part, related to the disappointing clinical translation and efficacy of tissue engineering technologies and should be considered in their design. Classical regenerative medicine strategies utilize stem cells, growth factors and biomaterials alone or in combination to promote tissue development¹¹. More recently, the role of the immune system in tissue repair is being recognized as a central factor in determining healing outcomes leading to the introduction of immunomodulation as a new therapeutic modality in regenerative medicine technology design. However, there are also numerous age-related changes that occur in the immune system, termed inflammaging, that may impede a regenerative therapeutic response¹². Age-related immune changes have been primarily studied in the context of infectious disease, chronic inflammatory conditions, vaccine efficacy and more recently cancer immunotherapy efficacy but may also negatively impact the response to tissue damage and regenerative immunotherapies¹³. For example, T cell numbers decrease with aging and there is a myeloid shift in the bone marrow^{14,15}. Furthermore, there are composition changes in the T cell compartment with aging that include increased CD8⁺ T cells, reduced naïve CD4⁺ T cells, and increased effector CD4⁺ T cells which altogether may compromise tissue development¹⁴. Here, we investigated how immunological changes associated with aging impact the response to muscle injury and limit the regenerative capacity of a therapeutic biological scaffold. Targeting age-associated immunological changes that inhibit a regenerative response may enable recovery of a therapeutic response and restoration of tissue repair capacity in older organisms.

### SUMMARY

In one embodiment, the present disclosure relates to a method for promoting wound healing, tissue repair, tissue regeneration or any combination thereof, in a subject in need thereof. Specifically, the method comprises administering a therapeutically effective amount of at least one pharmaceutical composition comprising at least one IL-17 antagonist and at least one regenerative therapy to the subject. In the above method, the at least one IL-17 antagonist and at least one regenerative therapy are administered simultaneously to the subject. In another aspect of the above method, at least one IL-17 antagonist and at least one regenerative therapy are administered sequentially to the subject. In another aspect, the IL-17 antagonist, regenerative therapy, or the IL-antagonist and regenerative therapy are administered systemically to the subject. In still yet another aspect, the at least one IL-17 antagonist, at least one regenerative therapy, or the at least one IL-17 antagonist and at least one regenerative therapy are administered locally to the site of the wound or area of tissue repair or regeneration in the subject.

In the above method, the subject in need of treatment thereof can have one or more inhibitory factors that inhibit or prevent regeneration. More specifically, the inhibitory factors may be age, infection, autoimmune disease, or any combination thereof.

In yet other aspects, in the above method, the IL-17 antagonist is an IL-17 antibody or an antigen-binding portion thereof. In still further aspects, the IL-17 antibody, or antigen-binding portion thereof, is a monoclonal antibody, a chimeric antibody, a bi-specific antibody, a human antibody, or antigen-binding portion thereof. In still further aspects, the IL-17 antibody, or antigen-binding portion thereof, is a human antibody. More specifically, in yet further aspects, the human antibody, or antigen-binding portion thereof, can specifically bind to human IL-17A, human 1L-17F and/or human IL-17A/F.

In other aspects of the above method, the at least one regenerative therapy is stem cells, platelet-rich plasma, extracellular matrix (ECM), prolotherapy, lipogems, or any combinations thereof.

In yet other aspects of the above method, the method further comprises a single pharmaceutical composition containing at least one IL-17 antagonist and at least one regenerative therapy.

In some aspects of the above method, the pharmaceutical composition is a delayed-release or sustained-release composition.

In some aspects of the above method, the method further comprises a first pharmaceutical composition containing at least one IL-17 antagonist and a second pharmaceutical composition containing at least one regenerative therapy.

In another embodiment, the present disclosure relates to a kit for use in promoting wound healing, tissue repair, tissue regeneration, or any combination thereof, in a subject in need thereof. In one aspect, the method comprises at least one pharmaceutical composition comprising at least one IL-17 antagonist and at least one regenerative therapy.

In one aspect, IL-17 antagonist in the kit is an IL-17 antibody or an antigen-binding portion thereof. More specifically, in another aspect, the IL-17 antibody, or antigen-binding portion thereof, is a monoclonal antibody, a chimeric antibody, a bi-specific antibody, a human antibody, or antigen-binding portion thereof. In still further aspects, the IL-17 antibody, or antigen-binding portion thereof, is a human antibody.

In yet another aspect, the at least one regenerative therapy in the kit is stem cells, platelet-rich plasma, extracellular matrix (ECM), prolotherapy, lipogems, or any combinations thereof.

In still a further aspect, the kit comprises a single pharmaceutical composition containing at least one IL-17 antagonist and at least one regenerative therapy. In some aspects, the pharmaceutical composition is a delayed-release or sustained release composition.

In further aspects, the kit comprises a first pharmaceutical composition containing at least one IL-17 antagonist and a second pharmaceutical composition containing at least one regenerative therapy. In still further aspects, the first pharmaceutical composition, the second pharmaceutical composition or both the first pharmaceutical composition and the second pharmaceutical composition can be a delayed-release or sustained release composition.

### BRIEF DESCRIPTION OF THE FIGURES

FIG. 1 shows that aging alters immune-stromal response to the regenerative ECM biomaterials and impairs immune-stromal communication in muscle. a, Schematic illustration of experimental design including no injury control (N), volumetric muscle loss injury treated with saline (S) and VML treated with ECM in young (6 wk) and old (72 wk). b, Quantification of Th2 or Th17-related genes in muscle 1 week after injury or ECM treatment (n=3 young; n=3-4 old)._c, Quantification of immune cells from spectral flow cytometry with significant population changes between young and old mice with muscle injury and treatment (n=5). Eosinophils are presented as % of CD11b⁺, NKTs % of CD3⁺γδ⁻, and CD4 or CD8% of CD3⁺γδ⁻NK⁻. d, Transverse section of the quadricep muscle 1 week after injury or ECM stained with H&E. The black arrow indicates the ectopic adipogenesis region, and the dotted line demonstrates immune cell infiltrated area. e, UMAP overview of cell clusters identified using scRNA-seq data set on muscle from young and aged animals with injury or treatment (3 mice pooled for each condition). f, Age-specific global signaling network (top) and heatmap of predicted cluster-cluster signaling for selected clusters determined by Domino (bottom). The values shown are the summed z-scored expression values for ligands (in the ligand-cluster) targeting receptors predicted to be activated in the receptor-cluster. Higher values indicate increased expression of ligands predicted to be active for a given receptor cluster. g, NMF-CoGAPS analysis of fibroblast populations (top) and myeloid/macrophage populations (bottom). Region of cells expressing high levels of the gene sets are circled. Two-way ANOVA with Tukey's multiple comparisons test in (b-c). p values in NMF-CoGAPS were determined using Mann-Whitney U test and adjusted with false discovery rate correction for multiple testing in (g). *p<0.05, **p<0.01, and ****p<0.0001. For all bar graphs, data are mean±s.d.

FIG. 2 shows that Aging induces a Th17-associated transcription network and aged CD4 T cells demonstrate increased Th17 skewing and a unique secretome. a, Venn diagram illustrating shared or age-specific receptors and TFs (left), and protein-protein interaction network for the TFs specific to aged animals using STRING network (right). b, Gene set enrichment analysis using Enrichr of age-specific TFs in muscle. Adjusted p value (log 10) of significant GO terms are shown. c, Volcano plot of genes expressed in aged lymph node normalized to those in young lymph node (top left). Gene scoring for helper T cell pathways (top right) or differentially expressed genes for NF-κb/TNFα or Th17-associated pathways (bottom) based on Nanostring analysis (n=6). d, Multiparametric flow cytometry quantification of IFNγ⁺ or IL17A⁺ CD4 or γδ T cells in the lymph nodes from young or aged animals without injury or treatment (n=9 CD4 T; n=6 γδ T). e, Quantification (left) and representative plots (right) of flow cytometry analysis on CD4 T cells isolated from lymph nodes or spleens of young and old animals. Naïve phenotype (CD4+CD44⁻CD62L⁻), effector phenotype (CD4⁺CD44⁺CD62L⁻) and Th17 cells (CD4⁺CD44⁺CD62L⁻RORγt⁺) are shown (n=3). f, Schematic illustration of naïve T cell isolation and Th17 differentiation in vitro (left) and quantification of flow cytometry analysis on the undifferentiated and differentiated CD4 T cells (right; n=3). g, Quantification (left) and representative images (right) of the proteome profiler performed on cell culture supernatant from Th17-differentiated CD4 T cells from young and old mice (3 samples pooled for each condition). Protein molecules with significant differences in pixel densities compared to young animals are labeled and quantified using iBright Analysis Software. Unpaired two-tailed t-test (d-e). Two-way ANOVA with Tukey's multiple comparisons test (f). For all bar graphs, data are mean±s.e.m (d) or s.d. (e-f).

FIG. 3 shows that injury and ECM treatment in aged animals promote local and systemic IL17-associated immune response that inhibits tissue repair. a, Schematic illustration of various immune environments that are affected by injury. b, Representative images of flow cytometry comparing IL17A+ γδ T cells between young and aged animals (left) and the quantification of IL17 producing cell subtypes in muscle 1 week after injury or ECM treatment (n=3-4); γδ T cell, CD4 T cell, and innate lymphoid cell (ILC). c-f, in lymph node 1 week after injury or ECM treatment. c, Representative images of the lymph nodes (top left) and the quantification of their size (top right). Scale bar, 2 mm. Transverse section of the lymph nodes is stained with H&E (bottom). d, Quantification of T helper cell cytokine-related genes in lymph node (n=3-5 young; n=3-4 old). e, Quantification of Th17-associated genes in the lymph nodes (n=3-5). Protein-protein interaction demonstrating their association with IL17A or IL17F are shown using STRING network (top left). f, Representative images of flow cytometry data comparing IL17A⁺ γδ T cells between young and aged animals (top) and the quantification of IL17 producing cell subtypes in the lymph node (n=3-4). g, Representative images (left) and quantitative analysis (right) of the proteome profiler performed on blood serum from young and aged mice without the treatments (3 mice pooled for each condition). Protein molecules with significant differences in pixel densities compared to young animals are labeled and quantified using imageJ. Two-way ANOVA with Tukey's multiple comparisons test (c-e), one-way ANOVA with Tukey's multiple comparisons test (b, f), unpaired two-tailed t-test (g). For all bar graphs, data are mean±s.d.

FIG. 4 shows that local IL17 suppression rejuvenates the type 2 immune response to injury and ECM to restore tissue repair and reduce fibrosis in old animals. a, Schematic illustration of experimental design (left) and quantification of flow cytometry data for IL4⁺ CD4 T cells and eosinophils in muscle 3 weeks after injury (right; n=6 no injury; n=8 isotype; n=3 αIL17A and αIL17F). b, Experimental schematics (left), and representative images flow cytometry showing IL4⁺CD45 or CD4 T cells (middle) and quantification of IL4⁺ cell populations in muscle 6 weeks after injury and ECM treatment (right; n=4). c, Quantification of genes associated with fibrosis or adipogenesis in muscle (top left; n=3), and transverse section of the quadricep muscle 6 weeks after injury stained with Masson's Trichrome. d, Immunofluorescence images of the quadricep muscle 6 weeks after injury stained with dystrophin (top) or laminin (bottom). Quantification of muscle fibers with central nuclei are shown (right; n=3). Nuclei were stained with DAPI (represented in yellow). One-way ANOVA with Tukey's multiple comparisons test (a-d). *p<0.05, **p<0.01, and ****p<0.0001. For all bar graphs, data are mean±s.e.m (a) or s.d. (b-d).

FIG. 5 shows a schematic representation of the gating strategy used to identify indicated cell phenotypes from single cell suspensions from mouse muscle tissue using spectral flow cytometry.

FIG. 6 shows the changes in immune and stromal cell phenotypes 1 week after injury or ECM treatment between young and aged animals. a, Overview of UMAP plots from total live cells in muscle showing the population of the indicated cell phenotypes. b, Quantification of immune-stromal cells in muscle after treatment as determined by flowcytometry. (a, b) Indicated cell population represents average value of n=5 per group.

FIG. 7 shows the flow cytometry quantification (cell counts) of the indicated immune and stromal cell phenotypes in muscle from young and aged animals 1 week after injury and treatment. Statistical analysis was performed using a one-way ANOVA with Tukey's multiple comparisons test within the respective age groups (n=5). *p<0.05, **p<0.01, ***p<0.001, and ****p<0.0001. For all bar graphs, data are mean±s.d.

FIG. 8 shows the flow cytometry quantification (cell percentages) of a, CD45+/CD45− cells or myeloid/lymphoid cells (indicated cell population represents average value of n=5 per group), and b, indicated immune/stromal cell phenotypes in muscle between young and aged animals 1 week after the treatments. Statistical analysis was performed using a one-way ANOVA with Tukey's multiple comparisons test within the respective age groups (n=5). *p<0.05, **p<0.01, ***p<0.001, and ****p<0.0001. For all bar graphs, data are mean±s.d.

FIG. 9 shows single cell RNA sequencing-based cell clustering information in young and aged muscle tissue 1 week after injury or ECM treatment. a, Heatmap of differentially expressed genes with highest log fold-change from each cluster (left) and UMAP plot with cluster labels and key signature genes (right). b, Signature gene markers for single cell clusters. Each dot shows the expression of genes associated with cluster identity. Gene expression after normalization to the maximum averaged expression are shown. c, Overview of cell clusters identified in UMAP plots based on the key gene expression.

FIG. 10 shows gene expressions of cluster-defining markers shown in violin plots.

FIG. 11 shows twelve top differentiated genes of each cluster population. Values represent log 10 fold change between each cluster compared to all other clusters.

FIG. 12 shows quantification of each cell cluster between the a, age groups or b, treatment groups using single cell RNA sequencing dataset.

FIG. 13 shows gene signature of fibroblast populations using NMF CoGAPS. p values are determined by Mann-Whitney U test and adjusted with false discovery rate correction for multiple testing.

FIG. 14 shows gene signature of myeloid/macrophage populations using NMF CoGAPS. p values are determined by Mann-Whitney U test and adjusted with false discovery rate correction for multiple testing.

FIG. 15 shows a full list of receptors and transcription factors that are common or specific to young or aged animals using Domino.

FIG. 16 shows heatmaps of activation score for young animal-specific transcription factors and their correlation with receptor expression. Transcription factors specific to young animals are labelled on the right, and the correlated receptors are labeled on top right.

FIG. 17 shows protein-protein interaction network (force-directed graph layout) for the transcription factors specific to young animals using STRING network.

FIG. 18 shows a list of significantly up- or down-regulated genes (p<0.05) in the aged lymph nodes compared to young lymph nodes from Nanostring analysis (n=6).

FIG. 19 shows pathway scoring of the genes associated with Th1-, Th2-, Th17-differentiation or Th17-mediated biology from Nanostring analysis (n=6).

FIG. 20 shows immune phenotype changes in the inguinal lymph node from young and aged animals without injury or ECM treatment. a, Representative images of flow cytometry data showing IFNγ⁺ or IL17A⁺ γδ or CD4 T cells in the lymph node. b, Representative images (left), and quantification of flow cytometry data showing γδ T cell population in CD3⁺ T cells in the lymph node (n=6). Unpaired two-tailed t-test (b). For all bar graphs, data are mean±s.e.m.

FIG. 21 shows quantification of inflammation- or senescence-associated genes in muscle 1 week after injury and treatment. Statistical analysis was performed using a two-way ANOVA with Tukey's multiple comparisons test (n=3-4). For all bar graphs, data are mean±s.d.

FIG. 22 shows representative images of flow cytometry data showing IL17A+CD4 T and innate lymphoid cells from young and aged animals (top) and the cell count quantification of IL17 producing cell subtypes in muscle 1 week after injury or ECM treatment (bottom). Statistical analysis was performed using a one-way ANOVA with Tukey's multiple comparisons test within the respective age groups (n=3-4). For all bar graphs, data are mean±s.d.

FIG. 23 shows whole image of transverse section of the quadricep muscle 1 week after injury or ECM treatment stained with H&E (top) or Masson's Trichrome (bottom).

FIG. 24 shows differential B cell response to ECM with aging. a, Cell count quantification of CD45⁺ immune cells in the lymph node 1 week after injury and treatment. b, Representative images of flow cytometry data showing CD3⁺CD19⁻ T cells or CD3⁻CD19⁺ B cells (left) and quantification of B cells and B/T cell ratio 1 week after injury or treatment (right). Statistical analysis was performed using a one-way ANOVA with Tukey's multiple comparisons test within the respective age groups (n=3-5). For all bar graphs, data are mean±s.d.

FIG. 25 shows representative images of flowcytometry data showing IL17A⁺ CD4 T or innate lymphoid cells between young and aged animals (top) and the cell percentage quantification of IL17 producing cell subtypes (bottom) in the lymph node 1 week after injury or treatment. Statistical analysis was performed using a one-way ANOVA with Tukey's multiple comparisons test within the respective age groups (n=3-4). For all bar graphs, data are mean±s.d.

FIG. 26 shows representative images (left) and quantitative analysis (right) of the proteome profiler array performed on blood serum from young and aged mice 1 week after injury. Protein molecules with significant differences in pixel densities between the groups are labeled and quantified using imageJ. Serum from 3 animals were pooled for analysis. Statistical analysis was performed using a Two-way ANOVA with Tukey's multiple comparisons test. *p<0.05, **p<0.01, and ***p<0.001. For all bar graphs, data are mean±s.d.

FIG. 27 shows immune-modulatory effect of αIL17 treatment. a, Schematic illustration of experimental design (left) and quantification of senescence- or inflammation-associated genes in muscle after various injection regime (right). b, Illustration of experimental design (left) and quantification of flow cytometry data showing the total number of CD45⁻ or CD45⁺ live cells or IL4⁺CD45⁺ immune cells in muscle 10 days after VML injury (right). Statistical analysis was performed using a one-way ANOVA with Tukey's multiple comparisons test (n=3-4). *p<0.05, and **p<0.01. For all bar graphs, data are mean±s.d.

FIG. 28 shows flow cytometry quantification of the indicated immune cell phenotypes in aged 4Get muscle 6 weeks after injury. Statistical analysis was performed using a one-way ANOVA with Tukey's multiple comparisons test (n=4). *p<0.05. For all bar graphs, data are mean±s.d.

FIG. 29 shows changes in immune and stromal cell phenotypes after αIL17 and ECM combination therapy in C57BL/6J aged animals. Tissues were analyzed 3 weeks after injury. Quantification of live cells in muscle showing the population of indicated cell phenotypes. Colored bar charts represent average cell count value of n=4-5. Statistical analysis was performed using a one-way ANOVA with Tukey's multiple comparisons test. *p<0.05, **p<0.01, ***p<0.001, and ****p<0.0001. For all bar graphs, data are mean±s.d.

FIG. 30 shows quantification of inflammation-, senescence-, adipose- or fibrosis-associated genes in aged C57BL/6J muscle 6 weeks after surgery. Statistical analysis was performed using a one-way ANOVA with Tukey's multiple comparisons test (n=3-4). *p<0.05, **p<0.01, ***p<0.001, and ****p<0.0001. For all bar graphs, data are mean±s.d.

FIG. 31 shows transverse section of the quadricep muscle 6 weeks after injury stained with Masson's Trichrome, dystrophin or laminin. Quantification of muscle fibers with central nuclei are shown. Statistical analysis was performed using a one-way ANOVA with Tukey's multiple comparisons test. For all bar graphs, data are mean±s.d.

FIG. 32 shows heatmaps of activation score for aged animal-specific transcription factors and their correlation with receptor expression. Transcription factors specific to aged animals are labelled on the right, and the correlated receptors are labeled on top right.

FIG. 33 shows heatmap of predicted cluster-cluster signaling. The values shown are the summed z-scored expression values for ligands (in the ligand-cluster; L_) targeting receptors predicted to be activated in the receptor-cluster (R_). Higher values indicate increased expression of ligands predicted to be active for a given receptor cluster.

FIG. 34 shows that aging induces a Th17-associated transcription network and impairs immune-stromal communication in muscle. Specifically, Age-specific global signaling network (top) and inter-cluster correlation determined by Domino from the scRNA-seq demonstrating the variety and strength of the communication interactions (bottom).

FIG. 35 shows that aging alters immune-stromal response to the regenerative ECM biomaterials. Specifically, quantification of immune cells from spectral flow cytometry with significant population changes between young and old mice with muscle injury and treatment (n=5). Eosinophils are presented as % of CD11b⁺, NKTs % of CD3⁺γδ⁻, and CD4 or CD8% of CD3⁺γδ⁻NK⁻.

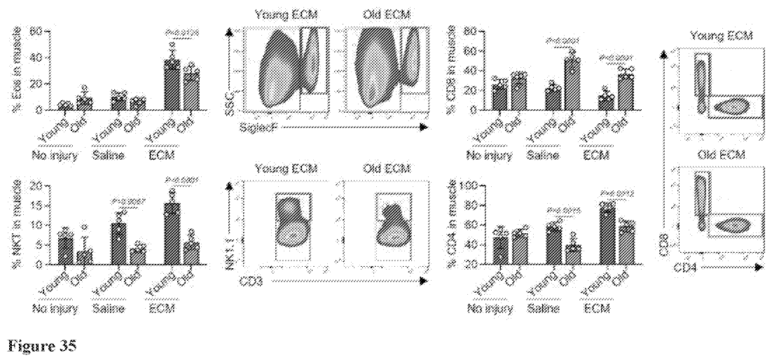

FIG. 36 shows that quantification of Th2 or Th17-related genes in muscle (n=3 young; n=3-4 old) 1 week after injury or ECM treatment.

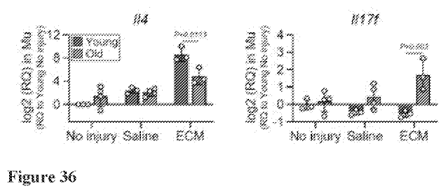

### DETAILED DESCRIPTION

### I. Definitions

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting.

As used herein, the term “about” or “approximately” means within an acceptable error range for the particular value as determined by one of ordinary skill in the art, which will depend in part on how the value is measured or determined, (i.e., the limitations of the measurement system). For example, “about” can mean within 1 or more than 1 standard deviations, per practice in the art. Where particular values are described in the application and claims, unless otherwise stated, the term “about” means within an acceptable error range for the particular value.

As used herein, the term “administering” in relation to a compound, e.g., an IL-17 inhibitor, is meant to refer to delivery of that compound by any route, including, for example, local administration at the site of inflammation or injury.

As used herein, the term, “extracellular matrix” or “ECM” refers to a scaffold in a cell's external environment with which the cell interacts via specific cell surface receptors. The extracellular matrix serves many functions, including, but not limited to, providing support and anchorage for cells, segregating one tissue from another tissue, and regulating intracellular communication. The extracellular matrix is composed of an interlocking mesh of fibrous proteins and glycosaminoglycans (GAGs). Examples of fibrous proteins found in the extracellular matrix include collagen, elastin, fibronectin, and laminin. Examples of GAGs found in the extracellular matrix include proteoglycans (e.g., heparin sulfate), chondroitin sulfate, keratin sulfate, and non-proteoglycan polysaccharide (e.g., hyaluronic acid). The term “proteoglycan” refers to a group of glycoproteins that contain a core protein to which is attached one or more glycosaminoglycans.

As used herein, the term “interleukin-17” (or “IL-17”) can include the IL-17 family of cytokines contains six members, IL-17 (also called IL-17A), IL-17B, IL-17C, IL-17D, IL-17E (also known as IL-25) and IL-17F or naturally occurring variants thereof. These polypeptides consist of 163-202 amino acids with molecular masses of 20-30 kDa. They share four conserved cysteine residues at C-terminal region that may participate in the formation of intermolecular disulfide linkages.

As used herein, an “IL-17 antagonist” is meant to refer to a molecule capable of antagonizing (e.g., reducing, inhibiting, decreasing, blocking, delaying) IL-17 activity, such as IL-17 function and/or signaling (e.g., by blocking the binding of IL-17 to the IL-17 receptor). Non-limiting examples of IL-17 antagonists include IL-17 binding molecules and IL-17 receptor binding molecules. In some embodiments, the IL-17 antagonist is one or more antibodies (including monoclonal antibodies, chimeric antibodies, bi-specific antibodies, human antibodies, or antigen binding portions thereof (e.g., F(ab′)2 and Fab fragments), antibody fragments, oligonucleotides, polynucleotides, antisense oligonucleotides, enzymes, gene editing agents, nucleases, peptides, polypeptides, small molecules, synthetic compounds, natural compounds or combinations thereof.

As used herein, “regenerative therapy” refers to use of certain cells, biomaterials, or other materials to stimulate repair mechanisms and/or restore function in damaged body tissues, muscles or organs. Examples of cells that can be used include stem cells (e.g., adipose stem cells, embryonic stem cells, hematopoietic stem cells, induced pluripotent stem cells, umbilical cord blood mesenchymal stem cells, etc.). Examples of biomaterials that can be used include extracellular matrix (ECM), platelet-rich plasma and combinations thereof. Other examples of regenerative therapy include prolotherapy, lipogems and combinations thereof.

### II. Methods for Promoting Wound Healing, Tissue Repair, or Tissue Regeneration

In one embodiment, the present disclosure relates to a method for promoting wound healing, tissue repair, or tissue regeneration in a subject in need thereof. In some aspects, the method involves promoting wound healing. In another aspect, the method involves promoting tissue repair. In yet another aspect, the method involves promoting tissue regeneration.

The methods of the present disclosure involve administering to a subject in need of treatment at least one pharmaceutical composition comprising at least one IL-17 antagonist and at least one regenerative therapy to the subject. In some aspects, the at least one IL-17 antagonist is at least one IL-17 antibody or antigen-binding portion thereof, and the at least one regenerative therapy is a biomaterial (e.g., ECM or platelet-rich plasma). In another aspect, the at least one IL-17 antagonist is at least one IL-17 antibody or antigen-binding portion thereof, and the at least one regenerative therapy is a cell, such as a stem cell. In yet another aspect, the at least one IL-17 antagonist is at least one IL-17 antibody or antigen-binding portion thereof and prolotherapy or lipogems.

In some aspects, the subject is a mammal such as a monkey, ape or human. In other aspects, the subject is a human. In further aspects, the subject is a human that has one or more inhibitory factors that inhibit or prevent regeneration. In some aspects, the inhibitory factors may be age, suffering from an infection and/or autoimmune disease. In some aspects, the inhibitory factor is age. Specifically, the subject is at least 40 years of age. In other aspects, the subject is at least 45 years of age. In still other aspects, the subject is at least 50 years of age. In still further aspects, the subject is at least 55 years of age. In yet further aspects, the subject is at least 60 years of age. In yet further aspects, the subject is at least 65 years of age. In yet further aspects, the subject is at least 70 years of age. In yet other aspects, the subject is at least 75 years of age. In still further aspects, the subject is at least 80 years of age. In yet further aspects, the subject is at least 85 years of age. In yet further aspects, the subject is at least 90 years of age.

### III. Pharmaceutical Compositions

Provided herein are pharmaceutical compositions that comprise at least one IL-17 antagonist and at least one regenerative therapy and optionally, at least one pharmaceutically acceptable excipient, which may also be called a pharmaceutically suitable excipient or carrier (i.e., a non-toxic material that does not interfere with the activity of the active ingredient). A pharmaceutical composition may be a sterile aqueous or non-aqueous solution, suspension, gel or emulsion (e.g., a microemulsion). The excipients described herein are examples and are in no way limiting. An effective amount or therapeutically effective amount refers to an amount of the one or more IL-17 antagonists and one or more regenerative therapies administered to a subject, either simultaneously or sequentially, and either as a single dose or separate doses as well as part of a series of doses, which is effective to produce a desired therapeutic effect.

When one or more IL-17 antagonists and one or more regenerative therapies are administered to a subject for treatment of a disease or disorder described herein (e.g., to promote wound healing, tissue repair and/or tissue regeneration), the one or more IL-17 antagonists and one or more regenerative therapies may or may not be formulated into separate pharmaceutical compositions. A pharmaceutical preparation may be prepared that comprises each of the separate pharmaceutical compositions (which may be referred to for convenience, for example, as a first pharmaceutical composition and a second pharmaceutical composition comprising each of at least one IL-17 antagonist and at least one regenerative therapy, respectively). Each of the pharmaceutical compositions in the preparation may be administered at the same time (i.e., concurrently or simultaneously) and via the same route of administration or may be administered at different times (e.g., sequentially) by the same or different administration routes. Alternatively, one or more IL-17 antagonists and one or more regenerative therapies may be formulated together in a single pharmaceutical composition. For example, in some aspects, the single pharmaceutical composition may contain one or more particles.

In other embodiments, a combination of at least one IL-17 antagonist, at least one regenerative therapy, and at least one additional biologically active agent may be administered to a subject in need thereof. When at least one IL-17 antagonist, at least one regenerative therapy, and an additional agent are used together in the methods described herein (e.g., to promote wound healing, tissue repair and/or tissue regeneration), each of the agents may or may not be formulated into the same pharmaceutical composition or formulated in separate pharmaceutical compositions. A pharmaceutical preparation may be prepared that comprises each of the separate pharmaceutical compositions, which may be referred to for convenience, for example, as a first pharmaceutical composition, a second pharmaceutical composition and a third pharmaceutical composition comprising each of the IL-17 antagonist, regenerative therapy, and the additional agent, respectively. Each of the pharmaceutical compositions in the preparation may be administered at the same time and via the same route of administration or may be administered at different times by the same or different administration routes.

For example, antibodies, e.g., antibodies to IL-17, are typically formulated either in aqueous form ready for parenteral administration or as lyophilisate for reconstitution with a suitable diluent prior to administration. According to some embodiments of the disclosed methods and uses, the IL-17 antagonist, e.g., IL-17 antibody, is formulated as a lyophilisate. Suitable lyophilisate formulations can be reconstituted in a small liquid volume (e.g., 2 ml or less) to allow subcutaneous administration and can provide solutions with low levels of antibody aggregation. The use of antibodies as the active ingredient of pharmaceuticals is now widespread, including the products HERCEPTIN (trastuzumab), RITUXAN (rituximab), SYNAGIS (palivizumab), etc. Techniques for purification of antibodies to a pharmaceutical grade are well known in the art. When a therapeutically effective amount of an IL-17 antagonist, e.g., IL-17 binding molecules (e.g., IL-17 antibody or antigen-binding fragment thereof) or IL-17 receptor binding molecules (e.g., IL-17 antibody or antigen-binding fragment thereof) is administered by intravenous, cutaneous or subcutaneous injection, the IL-17 antagonist will be in the form of a pyrogen-free, parenterally acceptable solution. A pharmaceutical composition for intravenous, cutaneous, or subcutaneous injection may contain, in addition to the IL-17 antagonist, an isotonic vehicle such as sodium chloride, Ringer's solution, dextrose, dextrose and sodium chloride, lactated Ringer's solution, or other vehicles as known in the art.

Subjects may generally be monitored for therapeutic effectiveness using assays and methods suitable for the condition being treated, which assays will be familiar to those having ordinary skill in the art and are described herein. Pharmacokinetics of an IL-17 antagonist (or one or more metabolites thereof) that is administered to a subject may be monitored by determining the level of the IL-17 antagonist in a biological fluid, for example, in the blood, blood fraction (e.g., serum), and/or in the urine, and/or other biological sample or biological tissue from the subject. Any method practiced in the art and described herein to detect the agent may be used to measure the level of the IL-17 antagonist during a treatment course. The dose of an IL-17 antagonist described herein for treating a wound or promoting or improving tissue repair may depend upon the subject's condition, that is, stage of the disease, severity of symptoms caused by the disease, general health status, as well as age, gender, and weight, and other factors apparent to a person skilled in the medical art. Pharmaceutical compositions may be administered in a manner appropriate to the disease to be treated as determined by persons skilled in the medical arts. In addition to the factors described herein and above related to use of one or more IL-17 antagonists and regenerative therapies for promoting or improving wound healing, tissue repair or tissue regeneration, suitable duration and frequency of administration of one or more IL-17 antagonists and one or more regenerative therapies may also be determined or adjusted by such factors as the condition of the subject, the type and severity of the subject's disease, the particular form of the active ingredient, and the method of administration. Optimal doses of an agent may generally be determined using experimental models and/or clinical trials. The optimal dose may depend upon the body mass, weight, or blood volume of the subject. The use of the minimum dose that is sufficient to provide effective therapy is usually preferred. Design and execution of pre-clinical and clinical studies for an IL-17 antagonist (including when administered for prophylactic benefit) and regenerative therapy described herein are well within the skill of a person skilled in the relevant art. When one or more IL-17 antagonists and one or more regenerative therapies are administered to promote or improve wound healing and tissue repair, the optimal dose of each IL-17 antagonist agent and regenerative therapy may be different.

An amount of an IL-17 antagonist that may be administered per day may be, not limited to, for example, between about 0.01 mg/kg and 100 mg/kg (e.g., between about 0.1 to 1 mg/kg, between about 1 to 10 mg/kg, between about 10-50 mg/kg, between about 50-100 mg/kg body weight. In other embodiments, the amount of an IL-17 antagonist that may be administered per day is between about 0.01 mg/kg and 1000 mg/kg, between about 100-500 mg/kg, or between about 500-1000 mg/kg body weight).

The optimal dose (per day or per course of treatment) may be different for the disease or disorder to be treated and may also vary with the administrative route and therapeutic regimen.

Pharmaceutical compositions comprising one or more IL-17 antagonists and/or one or more regenerative therapies can be formulated in a manner appropriate for the delivery method by using techniques routinely practiced in the art. The composition may be in the form of a solid (e.g., tablet, capsule), semi-solid (e.g., gel), liquid, or gas (aerosol). In other certain specific embodiments, the one or more IL-17 antagonists and/or one or more regenerative therapies (or pharmaceutical composition comprising same) is administered as a bolus infusion. In other certain specific embodiments, the one or more IL-17 antagonists and/or one or more regenerative therapies (or pharmaceutical composition comprising same) is administered as an implant, as described below.

Pharmaceutical acceptable excipients are well known in the pharmaceutical art and described, for example, in Rowe et al., Handbook of Pharmaceutical Excipients: A Comprehensive Guide to Uses, Properties, and Safety, 5^{th} Ed., 2006, and in Remington: The Science and Practice of Pharmacy (Gennaro, 21.sup.st Ed. Mack Pub. Co., Easton, Pa. (2005)). Exemplary pharmaceutically acceptable excipients include sterile saline and phosphate buffered saline at physiological pH. Preservatives, stabilizers, dyes, buffers, and the like may be provided in the pharmaceutical composition. In addition, antioxidants and suspending agents may also be used. In general, the type of excipient is selected based on the mode of administration, as well as the chemical composition of the active ingredient(s). Alternatively, compositions described herein may be formulated as a lyophilizate. A composition described herein may be lyophilized or otherwise formulated as a lyophilized product using one or more appropriate excipient solutions for solubilizing and/or diluting the agent(s) of the composition upon administration. Pharmaceutical compositions may be formulated for any appropriate manner of administration described herein as well as known in the art.

A pharmaceutical composition may be delivered to a subject in need thereof by any one of several routes known to a person skilled in the art. By way of non-limiting example, the composition may be delivered orally, intravenously, intraperitoneally, by infusion (e.g., a bolus infusion), subcutaneously, enteral, rectal, intranasal, by inhalation, buccal, sublingual, intramuscular, transdermal, intradermal, topically, intraocular, vaginal, rectal, or by intracranial injection, or any combination thereof. In certain particular embodiments, administration of a dose, as described above, is via intravenous, intraperitoneal, directly into the target tissue, joint space, or organ, or subcutaneous route. Formulations suitable for such delivery methods are described in greater detail herein.

According to some embodiments, one or more IL-17 antagonists and/or one or more regenerative therapies (which may be combined with at least one pharmaceutically acceptable excipient to form a pharmaceutical composition) is administered directly to the target tissue or site in need of treatment thereof. According to some embodiments, one or more IL-17 antagonists and/or one or more regenerative therapies (which may be combined with at least one pharmaceutically acceptable excipient to form a pharmaceutical composition) is administered locally, such as to the site of the wound or area of tissue repair or regeneration in the subject.

According to some embodiments, the one or more IL-17 antagonists and one or more regenerative therapies or pharmaceutical composition comprising the one or more IL-17 antagonists and/or one or more regenerative therapies may be formulated as a timed release (also called sustained release, controlled release) composition. Controlled or sustained release formulations can be achieved by the addition of time-release additives, such as polymeric structures, matrices, that are available in the art. A hydrogel formulation may be used to provide controlled or sustained release of one or more IL-17 antagonists and/or one or more regenerative therapies at the site of administration. Hydrogels are three-dimensional networks made of hydrophilic polymers or polymers containing hydrophilic co-polymers. Hydrogel networks are formed by the crosslinking of polymer chains via covalent bonds, hydrogen bonds, or ionic interactions, or via physical entanglement. Hydrogels can be prepared with biocompatible synthetic materials to achieve specific properties at the micro- or nanoscale level. The manipulation of the molecular weight or molecular weight distribution can be used to modulate the mechanical strength of hydrogels to satisfy different requirements. Hydrogels can be designed to modulate the porosity of the network, which can be advantageously used to control the release rate in conjunction with affinity of nucleic acid aptamers. Hydrogels can be designed in a wide variety of shapes as desired. Depending on the requirements, hydrogels can be prepared in different format of geometry such as particles, films, coatings, cylinders and slabs for in vitro and/or in vivo uses. Hydrogels can be formed from a wide variety of biocompatible polymeric materials, including, but not limited to, polyurethane, silicone, copolymers of silicone and polyurethane, polyolefins such as polyisobutylene and polyisoprene, nitrile, neoprene, collagen, alginate and the like. For example, suitable hydrogels can be formed from polyvinyl alcohol, acrylamides such as polyacrylic acid and poly(acrylonitrile-acrylic acid), polyurethanes, polyethylene glycol, poly(N-vinyl-2-pyrrolidone), acrylates such as poly(2-hydroxy ethyl methacrylate) and copolymers of acrylates with N-vinyl pyrrolidone, N-vinyl lactams, a poly (lactide-co-glycolide), acrylamide, polyurethanes, polyacrylonitrile, poloxamer, N-Isopropylacrylamide copolymers, poly(N-i sopropylacrylamide), poly(vinyl methyl ether), poly(NIPAAm-co-PEG) and the like.

Hydrogels can be prepared with natural biomolecules. For example, suitable natural hydrogels can be formed from gelatin, agarose, amylase, amylopectin, cellulose derivatives such as methylcellulose, hyaluronan, chitosan, carrangenans, collagen, Gellan™, alginate and other naturally derived polymers. For example, collagen can be used to form hydrogel. Collagen can be used to create an artificial extracellular matrix that can be used as cell infiltration scaffolds for inducing tissue regeneration and remodeling. Suitable natural hydrogels also include alginate. Alginate is natural polysaccharide extracted from algae or produced by bacteria. In another embodiment, agarose can be used to form a hydrogel.

A polymer formulation can also be utilized to provide controlled or sustained release of one or more IL-17 antagonists and/or regenerative therapies at the site of administration. Bioadhesive polymers described in the art may be used. By way of example, a sustained-release gel and the compound may be incorporated in a polymeric matrix, such as a hydrophobic polymer matrix. Examples of a polymeric matrix include a microparticle. The microparticles can be microspheres, and the core may be of a different material than the polymeric shell. Alternatively, the polymer may be cast as a thin slab or film, a powder produced by grinding or other standard techniques, or a gel such as a hydrogel. The polymer can also be in the form of a coating or part of a bandage, stent, catheter, vascular graft, or other device. The matrices can be formed by solvent evaporation, spray drying, solvent extraction and other methods known to those skilled in the art.

According to some embodiments, the compositions are formulated such that the one or more IL-17 antagonists and one or more regenerative therapies are bioavailable over an extended period of time following administration. According to some embodiments, the one or more IL-17 antagonists and one or more regenerative therapies maintain a concentration within a therapeutic window for a desired period of time.

In some embodiments, the compositions are formulated to bind to the affected tissues upon administration, and releasing the IL-17 antagonists and regenerative therapies and possible additional active agents over an extended period of time.

A pharmaceutical composition (e.g., for injection, IA injection, infusion, subcutaneous delivery, intramuscular delivery, intraperitoneal delivery or other method) may be in the form of a liquid. A liquid pharmaceutical composition may include, for example, one or more of the following: a sterile diluent such as water, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral composition can be enclosed in ampoules, disposable syringes or multiple dose vials made of glass or plastic. The use of physiological saline is preferred, and an injectable pharmaceutical composition is preferably sterile.

In certain embodiments, the pharmaceutical compositions comprising one or more IL-17 antagonists and/or one or more regenerative therapies are formulated for transdermal, intradermal, or topical administration. The compositions can be administered using a syringe, bandage, transdermal patch, insert, or syringe-like applicator, as a powder/talc or other solid, liquid, spray, aerosol, ointment, foam, cream, gel, paste. This preferably is in the form of a controlled release formulation or sustained release formulation administered topically or injected directly into the skin adjacent to or within the area to be treated (intradermally or subcutaneously). The active compositions can also be delivered via iontophoresis. Preservatives can be used to prevent the growth of fungi and other microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, thimerosal, and combinations thereof.

Pharmaceutical compositions comprising one or more IL-17 antagonists and/or one or more regenerative therapies can be formulated as emulsions for topical application. An emulsion contains one liquid distributed the body of a second liquid. The emulsion may be an oil-in-water emulsion or a water-in-oil emulsion. Either or both of the oil phase and the aqueous phase may contain one or more surfactants, emulsifiers, emulsion stabilizers, buffers, and other excipients. The oil phase may contain other oily pharmaceutically approved excipients. Suitable surfactants include, but are not limited to, anionic surfactants, non-ionic surfactants, cationic surfactants, and amphoteric surfactants. Compositions for topical application may also include at least one suitable suspending agent, antioxidant, chelating agent, emollient, or humectant.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. Liquid sprays may be delivered from pressurized packs, for example, via a specially shaped closure. Oil-in-water emulsions can also be used in the compositions, patches, bandages and articles. These systems are semisolid emulsions, micro-emulsions, or foam emulsion systems.

According to some embodiments, the one or more IL-17 antagonists and one or more regenerative therapies can be formulated with oleaginous bases or ointments to form a semisolid composition with a desired shape. In addition to the senolytic agent, these semisolid compositions can contain dissolved and/or suspended bactericidal agents, preservatives and/or a buffer system. A petrolatum component that may be included may be any paraffin ranging in viscosity from mineral oil that incorporates isobutylene, colloidal silica, or stearate salts to paraffin waxes. Absorption bases can be used with an oleaginous system. Additives may include cholesterol, lanolin (lanolin derivatives, beeswax, fatty alcohols, wool wax alcohols, low HLB (hydrophobellipophobe balance) emulsifiers, and assorted ionic and nonionic surfactants, singularly or in combination.

In accordance with some embodiments the present inventors contemplate use of specific HA binding peptides (HABPep) and extracellular matrix binding peptides (ECMBpep) which can recapture HA that is lost through a physical or biological mechanism and provide the stable anchor on the tissue surface that is necessary to dynamically bind and concentrate HA where it is needed. Such peptides are disclosed in WO2015/009787 and incorporated by reference herein.

The present disclosure provides biological polymers or microbeads wherein said biocompatible polymers comprise one or more IL-17 antagonists and one or more regenerative therapies and, potentially one or more additional active agents admixed therein, conjugated to one or more ECMBPep which are covalently linked to the biocompatible polymers; obtaining a sufficient amount of having one or more thiolated HA binding peptides (C-HABPep) in a suitable solution; adding the solution and mixing for a sufficient period of time to produce one or more biocompatible polymers having one or more HA binding peptides (HABPep) which are covalently linked to the biocompatible polymers which are covalently linked to one or more ECMBPep, and administering the solution into the site of tissue injury locally.

### IV. Kits

In another embodiment, the present disclosure relates to a kit for use in promoting wound healing, tissue repair, or tissue regeneration in a subject in need thereof. In one aspect, the kit comprises at least one pharmaceutical composition comprising at least one IL-17 antagonist and at least one regenerative therapy. In another aspect, the kit can comprise at least one first pharmaceutical composition comprising at least one IL-17 antagonist and at least one second pharmaceutical composition comprising at least one regenerative therapy. The kit can also contain instructions for using the at least one pharmaceutical composition.

In yet another aspect, the at least one IL-17 antagonist is at least one IL-17 antibody or an antigen-binding portion thereof. For example, the IL-17 antibody can be a monoclonal antibody, a chimeric antibody, a bi-specific antibody, a huma antibody or antigen-binding portion thereof. In some aspects, the IL-17 antibody is a human antibody. In some aspects, the at least one regenerative therapy is stem cells. In another aspect, the at least one regenerative therapy is a biomaterial (e.g., ECM, platelet-rich plasma or any combination thereof). In yet another aspect, the at least one regenerative therapy is prolotherapy, lipogems or any combination thereof. According to some embodiments, kits with unit doses of one or more of the agents described herein, usually in oral or injectable doses, are provided. Such kits may include a container containing the unit dose, an informational package insert describing the use and attendant benefits of the drugs in promoting or improving wound healing or tissue repair.

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges which may independently be included in the smaller ranges is also encompassed within the disclosure, subject to any specifically excluded limit in the stated range. Where the stated range includes one or both of the limits, ranges excluding both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and described the methods and/or materials in connection with which the publications are cited.

It must be noted that as used herein and in the appended claims, the singular forms “a”, “an”, and “the” include plural references unless the context clearly dictates otherwise. All technical and scientific terms used herein have the same meaning.

The publications discussed herein are provided solely for their disclosure prior to the filing date of the present application and each is incorporated by reference in its entirety. Nothing herein is to be construed as an admission that the present disclosure is not entitled to antedate such publication by virtue of prior disclosure. Further, the dates of publication provided may be different from the actual publication dates which may need to be independently confirmed.

### Example 1: Age-Related Immune-Stromal Networks Inhibit Response to Regenerative Immunotherapies

### Methods

### Surgical Procedures and Implantation

All animal procedures were approved by Johns Hopkins University Institutional Animal Care and Use Committee protocol. Mice aged 6 week (young) or 72 week (aged) were obtained from the Jackson Laboratory (C57BL/6J: stock #00064). 4Get mice (stock #004190) were obtained from the Jackson Laboratory and bred in-house. IL17A-GFP mice (courtesy of F. Housseau, Johns Hopkins, MD) were bred in-house. The bilateral muscle defects in quadricep were created as previously described⁸³. The defects were either filled with 0.05 cc of 200 mg/ml biomaterial scaffold. Decellularized porcine extracellular matrix (ECM) was used as a biological scaffold in 0.05 ml at a concentration of 200 mg/ml in phosphate-buffered saline (PBS). Control surgeries were treated with 0.05 ml of PBS. All materials were sterilized with UV before use. Immediately after surgery, mice were given subcutaneous injection of carprofen (Rimadyl, Zoetis) at 5 mg/kg for pain relief. For analysis, mice were euthanized at 1, 3, or 6 weeks after surgery, and various tissues (blood, inguinal lymph node, or muscle) were extracted. All animal procedures in this study were conducted in accordance with an approved Johns Hopkins University IACUC protocol.

### Tissue ECM Preparation

Porcine-derived tissues (Wagner Meats, Mt. Airy, MD) were processed following a protocol previously described⁸³. Tissues were formulated into a paste with particle sizes no larger than 5 mm² and rinsed thoroughly with distilled water. Tissues were then incubated in 3% peracetic acid (Sigma) on a shaker at 37° C. for 4 hours. pH was adjusted to 7 with running distilled water and PBS rinsing, and tested after solution was freshly changed. Samples were then transferred to a 1% Triton-X100 (Sigma)+2 mM sodium EDTA (Sigma) solution on a stir plate at 400 rpm, room temperature for 3 days. Tissues were then rinsed thoroughly with distilled water and incubated in 600 U/ml DNase I (Roche Diagnostics) for 24 hours. Tissues were rinsed with distilled water, frozen at −80° C. and lyophilized for at least 3 days. Finally, dry sample was turned into a particulate form using a SPEX SamplePrep Freezer/Mill (SPEX CertiPrep). ECM powder was stored in −20° C. until use, and UV sterilized immediately before use.

### Non-Surgical Animal Experiments

For experiments comparing base line immunological difference between young and aged, no surgery was given to young or aged animals, and blood or inguinal lymph node was analyzed using flow cytometry, qRT-PCR, proteome profiler and histological evaluation. Cytokine expression in blood was analyzed using proteome profiler cytokine array (R&D systems) according to the manufacturer's directions.

qRT-PCR

For total mRNA expression in muscle and inguinal lymph node, lysis was conducted on whole tissues using TRIzol at 1 week or 6 weeks after surgery. RNA purification was performed using RNeasy Plus Mini kit (Qiagen). PCR was all performed using TaqMan Gene Expression Master Mix (Applied Biosystems) according to the manufacturer's directions. Briefly, 2 ug of mRNA was synthesized into complementary DNA (cDNA) using Superscript IV VILO Master Mix (Thermo Fisher Scientific) and was used at 100 ng/well in a total volume of 20 μl of PCR. All qRT-PCRs were performed on the StepOnePlus Real-Time PCR System (Thermo Fisher Scientific). Rer1, OAZ1, and Hprt were used as the reference gene and experimental groups were normalized to either no surgery or saline-treated controls. Low-expressing mRNA transcripts were pre-amplified using the TaqMan Pre-Amp System (Thermo Fisher Scientific) following manufacturer's recommendations with 10 cycles of amplification.

### Flow Cytometry

Whole muscle or inguinal lymph node was harvested either without injury, 1, 3, or 6 weeks after surgery. Muscle tissues were obtained by cutting the quadriceps from the hip to the knee, finely diced and digested for 45 min at 37° C. with 1.67 Wunsch U/ml Liberase TL (Roche Diagnostics) and DNaseI (0.2 mg/ml; RocheDiagnostics) in RPMI 1640 medium (Gibco). The digested tissues were ground through 70 μm cell strainers (Thermo Fisher Scientific) and washed multiple times with PBS. For intracellular staining, cells were stimulated for 4 hrs with Cell Stimulation Cocktail plus protein transport inhibitors (eBioscience) diluted in RPMI 1640 medium supplemented with 10% fetal bovine serum (FBS). Cells were then washed and surface-stained, followed by fixation/permeabilization (Cytofix-Cytoperm, BD) and intracellular markers. Flow cytometry was performed using Attune NxT Flow Cytometer (Thermo Fisher Scientific) or Cytek Aurora (Cytek). Cells were stained with the antibody panels listed in Table 1.

TABLE 1
Antibody panel for the flow cytometry analysis.

| | | |
|---|---|---|
| Fluorophores | Antigen | Clone |

Antibodies used for spectral flow cytometry

| | | |
|---|---|---|
| BV421 | SiglecF | E502440 |
| SuperBright436 | CD19 | 1D3 |
| Pacific Blue | Ly6g | 1A8 |
| BV510 | CD31 | MEC13.3 |
| BV570 | CD45 | 30-F11 |
| BV605 | CD90.2 | 30-H12 |
| BV650 | Ly6c | HK1.4 |
| BV711 | GD | GL3 |
| BV750 | B220 | RA3-6B2 |
| BV785 | F4/80 | BM8 |
| BB515 | cKit | 2B8 |
| SparkBlue550 | CD3 | 17A2 |
| PerCP | MHCII IA/I-E | M5/114.15.2 |
| BB700 | CD8 | 53-6.7 |
| PerCP-eFluor710 | CD29 | Hmb1-1 |
| PE | CD115 | AFS98 |
| PE-Dazzle594 | CD11c | N418 |
| PE-Cy5.5 | CD68 | C68/684 |
| PE-Cy7 | CD200R3 | BA13 |
| AF647 | NK1.1 | PK136 |
| AF700 | CD11b | M1/70 |
| Zombie NIR | Viability | N/A |
| APC-Fire750 | CD36 | HM36 |
| APC-Fire810 | CD4 | GK1.5 |

Antibodies used for 4Get mice

| | | |
|---|---|---|
| GFP | IL4 | N/A |
| PerCP-Cy5.5 | CD3 | 1742 |
| PE-Cy7 | F4/80 | BM8 |
| PE594 | SiglecF | E502440 |
| PE | CD8 | 53-6 7 |
| APC | CD4 | GK1 5 |
| AF700 | CD11b | M1/70 |
| BV605 | CD45 | 30-F11 |
| BV510 | Ly6c | HK1.4 |
| Pacific Blue | Ly6g | 1A8 |
| eFluor780 | Viability | N/A |

Antibodies used for IL17A-GFP mice

| | | |
|---|---|---|
| GFP | IL17A | N/A |
| PE-Cy7 | CD4 | GK1 5 |
| PE594 | GD | GL3 |
| PE | CD3 | 17A2 |
| BV605 | CD45 | 30-F11 |
| Pacific Blue | CD90.2 | 30-H12 |
| eFluor780 | Viability | N/A |

Antibodies used for intracellular staining

| | | |
|---|---|---|
| V500 | CD45 | 30-F11 |
| BV711 | CD8 | 53-6 7 |
| AF488 | CD3 | 17A2 |
| PerCP-Cy5.5 | CD19 | 6D5 |
| PE594 | GD | GL3 |
| PE-Cy7 | CD4 | GK1.5 |
| APC | IFNgamma | XMG1.2 |
| AF700 | IL17A | TC11-18H10.1 |
| eFluor780 | Viability | N/A |

Antibodies used for memory and/or Th17 staining

| | | |
|---|---|---|
| BV421 | Tbet | 04-46 |
| BV510 | CD45 | 30-F11 |
| BV650 | CD44 | IM7 |
| BV711 | CD4 | GK1.5 |
| SparkBlue550 | CD3 | 17A2 |
| PE | RORγt | Q31-378 |
| PE-Dazzle594 | CD62L | MEL-14 |
| APC-eFluor780 | CD49d | R1-2 |
| Zombie NIR | Viability | N/A |

### Splenocyte Isolation and Th17 Differentiation In Vitro

Spleens from young or aged mice were ground through 70 μm cell strainers (Thermo Fisher Scientific) and washed multiple times with PBS. Cells were then incubated with ACK lysing buffer (Thermo Fisher Scientific) in dark for 10 minutes for red blood cell lysis, followed by multiple PBS wash. The cells were then differentiated using CellXVivo mouse Th17 differentiation kit (R&D systems) for 5 days. For flow cytometry analysis, the cells were collected and stained for flow cytometry. For proteome analysis, the media was changed to T cell culture media (RPMI 1640 with 10% FBS, 1% Penicillin-Streptomycin, 1 mM Sodium Pyruvate, 10 mM HEPES, and 50 nM 2-Mercaptoethanol) on day 5, then cultured for additional 2 days. Supernatant was analyzed using proteome profiler cytokine array (R&D systems) according to the manufacturer's directions.

### IL17 Neutralization Treatment

Mice received 3 injections 20 μl intra-muscular injections of isotype control (rat IgG2a, R&D systems), anti-IL17a (100 μg/ml, R&D systems), anti-IL17f (100 μg/ml, R&D systems), or anti-IL17a and anti-IL 17f combined, every other day. All mice received treatments either at the day of surgery (for dosing experiment) or at 1 week after surgery (all other experiments), and were harvested at 3 or 6 weeks after surgery.

### NanoString Gene Expression Analysis

Inguinal lymph nodes from no treat mice were used to isolate mRNA for NanoString analysis. Gene expression was evaluated using the NanoString AutoImmune Profiling Panel (NanoString Technologies, Inc.). 100 ng of RNA was added to a probe-set mixture, and hybridized for 20 hours at 65° C. All samples were processed using a NanoString Prep Station under high sensitivity mode, and mRNA target transcripts were counted using the nCounter digital analyzer system (NanoString Technologies, Inc.). Data was analyzed using nSolver software.

### Histopathology

Tissues were harvested 1 or 6 weeks after surgery and fixed in 10% neutral buffered formalin for 48 hours. Tissues then underwent stepwise dehydration in EtOH, followed by xylenes, and embedded in paraffin. Tissue samples were sectioned as 6 μm slices, then stained for histopathological examination using Masson's Trichrome, hematoxylin and eosin, or immunofluorescence. Dystrophin and Laminin were stained using tyramide signal amplification method with Opal-570 (PerkinElmer, catalog no. FP1488001KT). Briefly, after blocking with bovine serum albumin for 1 hours, the primary antibody was incubated at room temperature for 30 min, followed by 10 min of incubation with horseradish peroxidase (HRP) polymer-conjugated secondary antibody, and 10 min of Opal. Slides were then counterstained with 4′,6-diamidino-2-phenylindole (DAPI) for 5 min before being mounted using DAKO mounting medium (Agilent, catalog no. S302380-2). Imaging of the histological samples was performed on a Zeiss Axio Imager A2 and Zeiss AxioVision software version 4.2. Immunofluorescent images were analyzed using ImageJ software.

### Collection of Single Cell Data Sets

Drop-seq, a single cell microfluidics encapsulation technique, was used to prepare libraries for CD45⁺ enriched cell populations isolated from mouse quadriceps 1 week after the treatments. For the CD45⁺ enriched populations, dead cells were removed using the Miltenyi Biotec Dead Cell Removal Kit followed by Miltenyi Biotec CD45 MicroBeads to separate CD45⁺ and CD45⁻ cells. After separation, an equal amount of CD45⁺ and CD45⁻ cells were pooled directly prior to input to Drop-seq. Drop-seq was run following the McCarroll Lab's December 2015 iteration of their published protocol available from their website (http://mccarrolllab.org/dropseq/).

### Data Preprocessing and Batch Effect Correction

Seurat was used for most processing steps where other software is not specified84. All cell counts were pruned of cells with UMI counts below 200, cells with more than 10% mitochondrial genes, and genes expressed in fewer than 0.1% of cells. We then normalized and scaled the data with regression on UMI count, G2M score, S score and percent mitochondrial genes and integrated the data with Seurat. We then calculated principle components using the top 2000 most variable genes. UMAP and shared nearest neighbor graph construction with subsequent Louvain clustering was then run on principle components.

### Cluster Composition by Condition

To assess cluster contribution, clusters from CD45⁺ and CD45⁻ cells were normalized separately to avoid slight differences in percent of CD45⁺ cells from enrichment by sample skewing normalization. For each sample, total number of cells by cluster were calculated and then normalized to the total of CD45⁺ or CD45⁻ cells in the dataset for the sample. The proportions of each sample were then averaged by condition to determine a condition-level average.

### Phenotypic Assignment of Clusters

Seurat's CellCycleScoring function was used to score cells based on expression of a subset of genes previously identified as associated with the G2M or S phase⁸⁵. Differential expression testing for clusters was run using Mann-Whitney U tests. Each cluster was compared against all other clusters. The resulting gene expression profiles were examined to determine cluster phenotype. In many cases, unique expression of marker genes was sufficient to determine cluster identity.

### Intercellular Signaling Networks

Domino was used to investigate potential signaling patterns between clusters of cells. Domino predicts activated transcription factors by cell using SCENIC⁸⁶ and then constructs a network connecting transcription factors, receptors, and ligands based on similar expression patterns. Default parameters for network construction were used. Networks were calculated for old and young samples individually and then compared to determine signaling components specific to each condition.

### CoGAPS Analysis

scCoGAPS⁸⁷ was used to perform non-negative matrix factorization (NMF) to identify 10 underlying patterns. Prior to NMF, mitochondrial and ribosomal genes were removed. It decomposes the data two matrices containing sets of low dimensional features, one of which called the pattern matrix contains a set of weights for each cell and the other called the amplitude matrix the corresponding weight for which each gene. The gene weights in the columns of the amplitude matrix indicate how much a gene contributes to the expression pattern identified by the corresponding row of the pattern matrix. The cell weights in the rows of the pattern matrix indicate how strongly a cell is enriched for the feature and can be used to identify cells with similar expression patterns to the feature. Finally, other cell labels (condition of origin, cluster label, etc.) can be compared with feature scores to identify how feature expressions change with respect to experimental variables such that even if there are no differences in cell clustering with ECM treatment or age, gene signatures can still vary significantly and provide functional insights. The resulting amplitude and pattern matrices were subsequently used to identify cells enriched by pattern and the genes driving patterns. Feature numbers were selected to maximize distinct expression signatures.

### Statistical Analysis

All analyses of qRT-PCR data used Livak method, where ΔΔCt values were calculated and reported as relative quantification values calculated by 2-ΔΔCt. Data are displayed as mean±s.d. Statistical analysis was performed using a one-way or two-way ANOVA with Tukey's corrections applied using GraphPad Prism v8, with statistical significance designated at p<0.05. All groups were compared to each other for multiple comparisons unless otherwise stated.

### Results

### Aging Reduces Type 2 Immune and Tissue Repair Responses to Regenerative Biomaterials

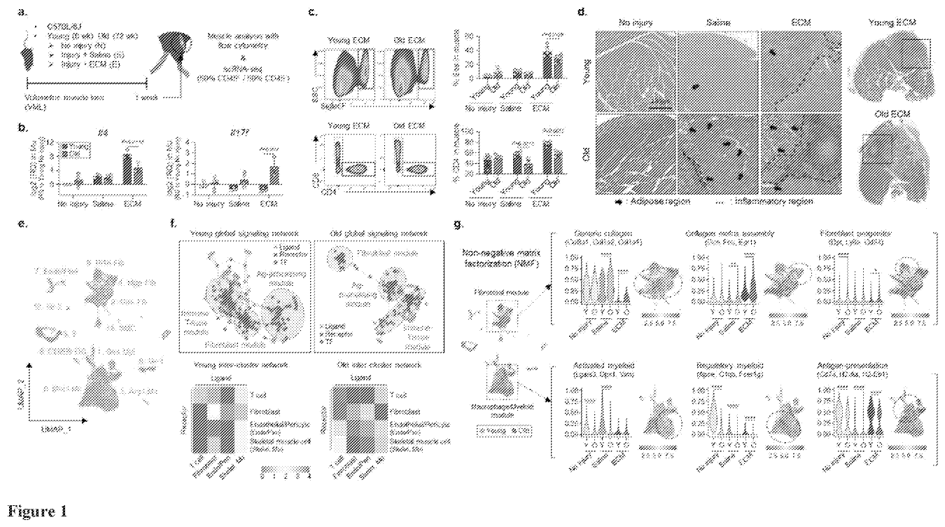

To evaluate the impact of aging on the immune response and resulting repair efficacy, we first characterized the response to an extracellular matrix (ECM) biomaterial in a muscle wound in young (6 week) and old (72 week) mice (FIG. 1a). ECM biomaterials, which can be derived from the matrix of different porcine and human tissue types¹⁶, are used for tissue repair in multiple clinical indications^{17,18} and are more easily delivered than cells and growth factors¹⁹⁻²¹. We utilized clinically available²² ECM from porcine small intestinal submucosa (SIS) as an example ECM material to model age-related differences in response. Muscle repair requires a type 2 immune_response with IL4 signaling^{23,24} and alternatively activated macrophages. Application of an ECM biomaterial in a muscle injury increases IL4 expression and promotes repair in part by increasing recruitment of IL-4 producing eosinophils and CD4⁺ T helper (Th) 2 cells²⁵.

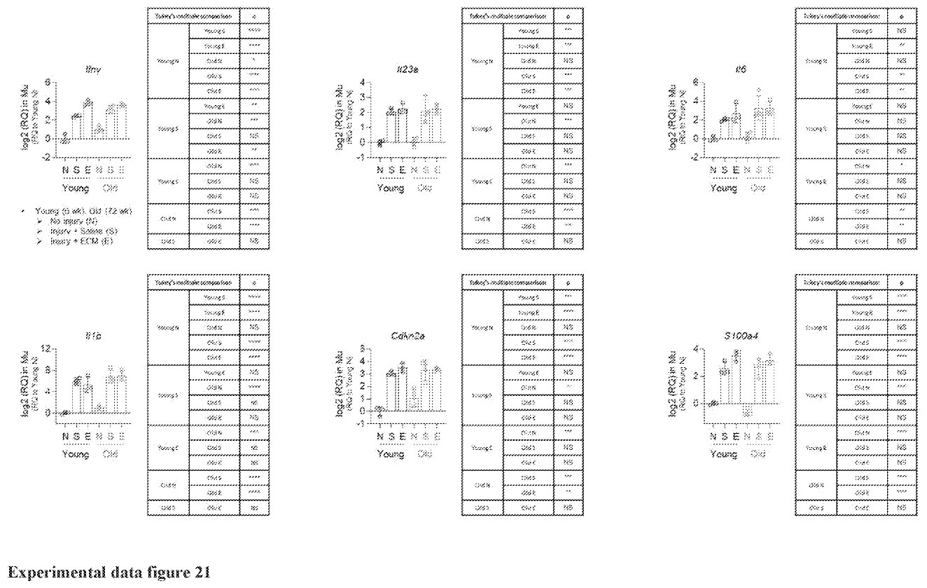

One week after volumetric muscle loss (VML) injury, aging significantly altered the cytokine gene expression profiles in injured tissue and with ECM treatment (FIG. 1b). In young animals, 114 gene expression increased in muscle tissue after injury with further significant increases after ECM treatment. However, 114 expression did not increase after injury in old mice and the level of expression after ECM treatment was significantly lower than the young animals. Instead, ECM treatment in aged mice significantly increased the expression of Il17f (Il17a gene expression was not detected in the muscle tissue of young or aged mice). Interestingly, other inflammatory genes, including Ifnγ, Il23a, Il6, Il1b, along with Cdkn2a and S100a4, all increased similarly in both young and old mice with injury or ECM treatment (FIG. 21).

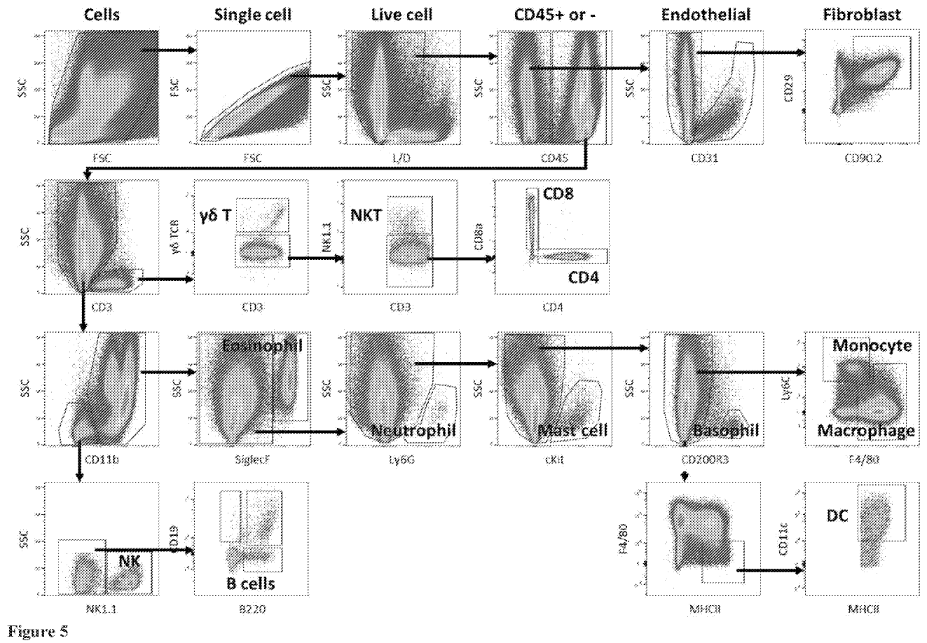

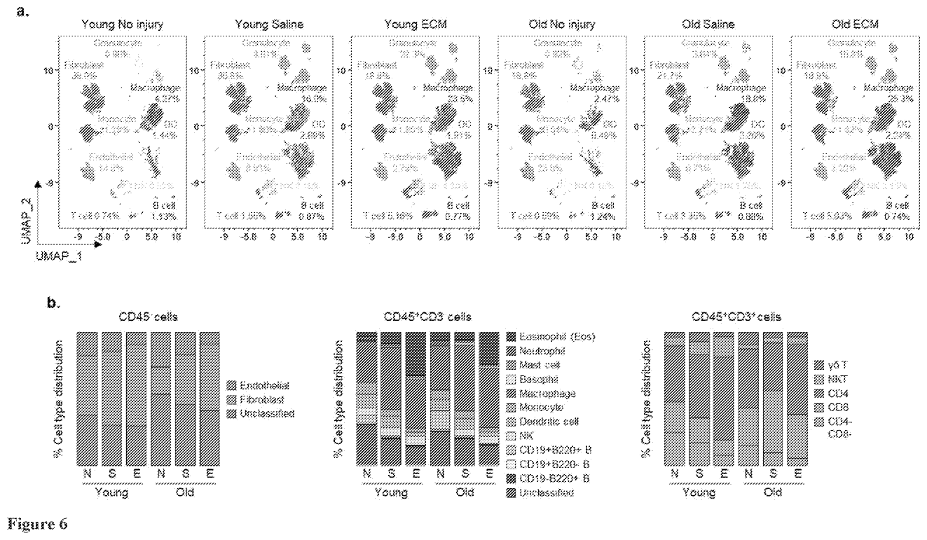

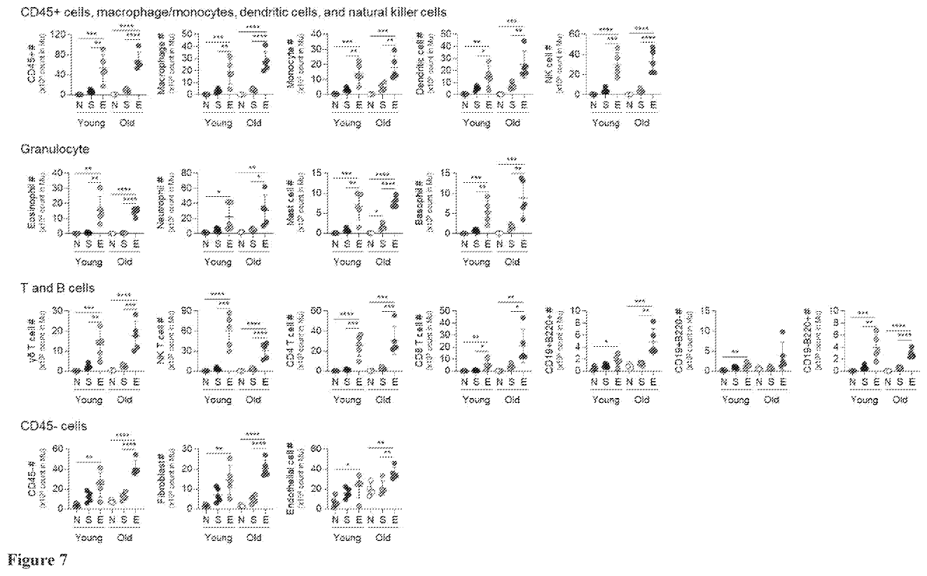

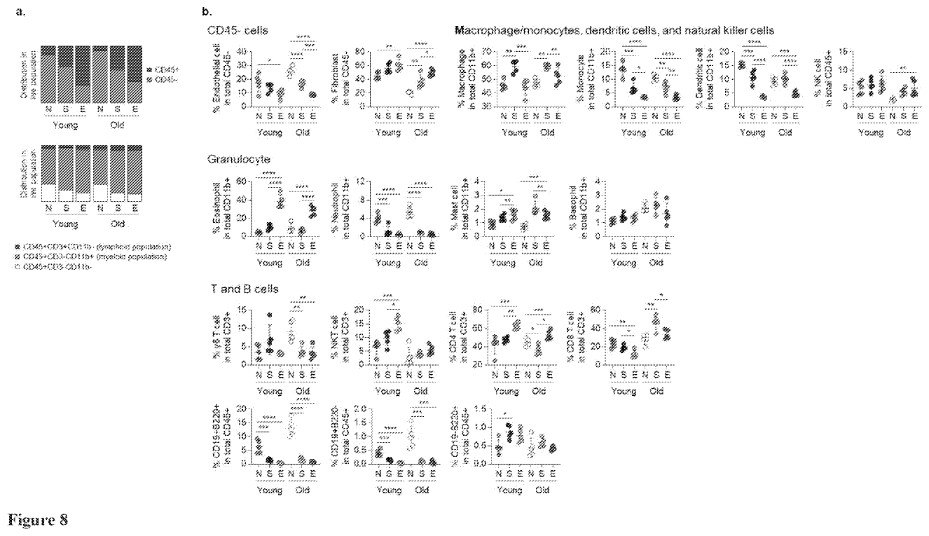

Next, using multiparametric spectral flow cytometry (FIG. 5), we found injury and ECM treatment resulted in distinct changes in immune and stromal cell responses in an aging tissue environment (FIG. 1c and FIG. 6-8). A robust cellular response to a volumetric muscle loss (VML) injury and ECM implant occurred in both young and aged animals but there were significant differences in the composition of the immune and stromal compartments. Specifically, the number and percentage of eosinophils in muscle tissue, a major source of regenerative type 2 response, significantly increased in young animals with injury and ECM treatment compared to no injury controls (FIG. 1c and FIG. 7-8). While the eosinophil counts and percentage also increased in the muscle injury of aged mice with ECM treatment (FIG. 7), their percentage as a total of all CD11b⁺ myeloid immune cells was significantly lower compared to young counterparts (p=0.0126; FIG. 8). The adaptive T cell immune response to muscle injury and ECM treatment also significantly changed with age with more CD8 T cells responding to injury in the aged animals compared to the CD4 and natural killer T (NKT) cell response in the young (FIG. 7). The CD45-population, including fibroblasts, endothelial and other stromal cells, also significantly increased in number after ECM treatment in aged animals compared to young animals (p=0.0057; FIG. 7).

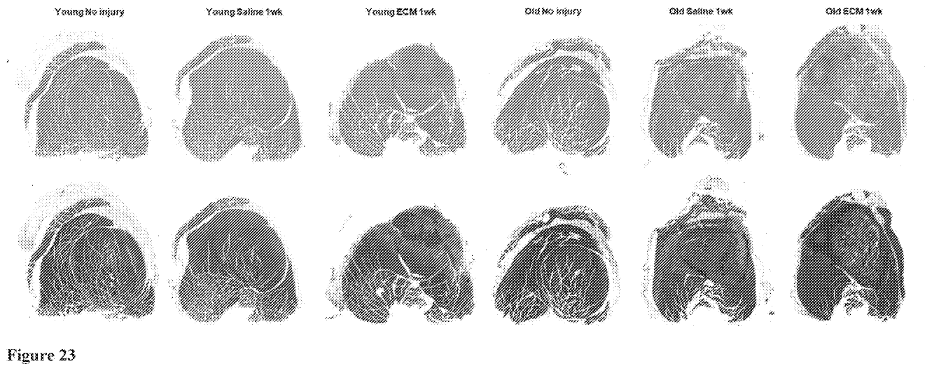

The significant differences in immune cell recruitment and cytokine expression correlated with changes in tissue repair observed histologically (FIG. 1d and FIG. 23). One week after injury and treatment, there was significant cell infiltration in both young and aged mice, with ECM treatment further increasing cellular infiltration and collagen deposition as visualized by Masson's Trichrome staining. The area of injury and ECM appeared much larger in the older animals suggesting that there was already significant repair occurring in young animals that did not occur in the aged tissue environment. There was also excessive adipose tissue in the muscle of aged animals compared to the younger counterparts, which increased further with ECM treatment (FIG. 1d).

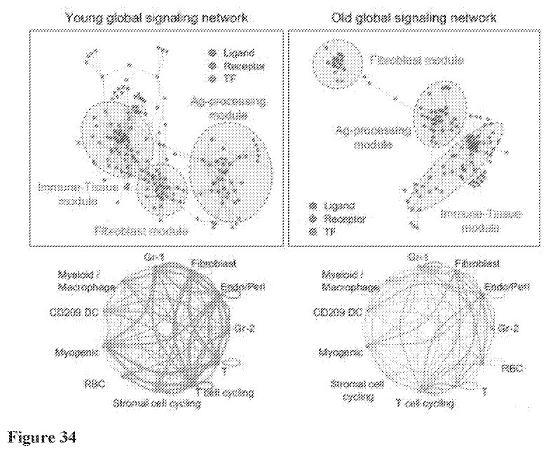

The adaptive T cell immune response to muscle injury and ECM treatment also significantly changed with age with more CD8 T cells responding to injury in the aged animals compared to the CD4 and natural killer T (NKT) cell response in the young (FIG. 34). Introduction of the ECM biomaterial further magnified the differences in T cell response producing a significant increase in CD4 and NKT cells in young mice and further increasing the CD8 T cells in aged mice (FIG. 7). The significant increase in CD4 T cell numbers in young animals combined with surge of CD8 T cells in the aged animals after ECM treatment resulted in a dramatic difference in the CD4 to CD8 ratio between young and aged mice that likely impacts the IL4 production required for muscle repair.

Additional changes in the injury and ECM response with aging occurred in the B cell and stromal cell populations. Before injury, the percentage of B cells was higher in aged muscle tissue (FIG. 8). After injury and migration of immune cells to the damaged tissue, the percentage of B cells was low in all groups, however, ECM treatment significantly increased the number of B cells, specifically the mature CD19⁺B220⁺ cells, only in the aged animals (FIG. 7). Finally, the CD45− population, including fibroblasts, endothelial and other stromal cells, significantly increased in number after treatment with ECM in aged animals compared to young animals (p<0.0057; FIG. 7).

Single Cell Analysis Reveals Age-Specific Immune and Stromal Response after Injury and Regenerative Medicine Treatment

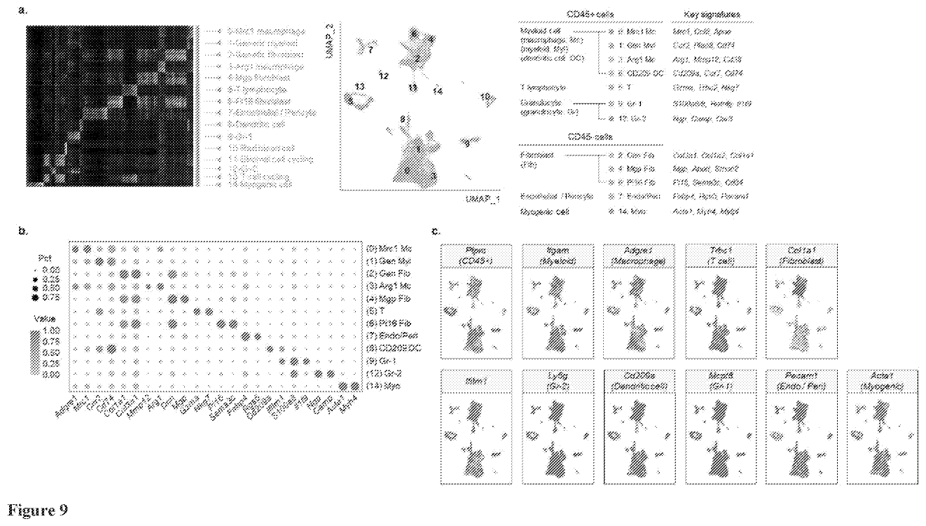

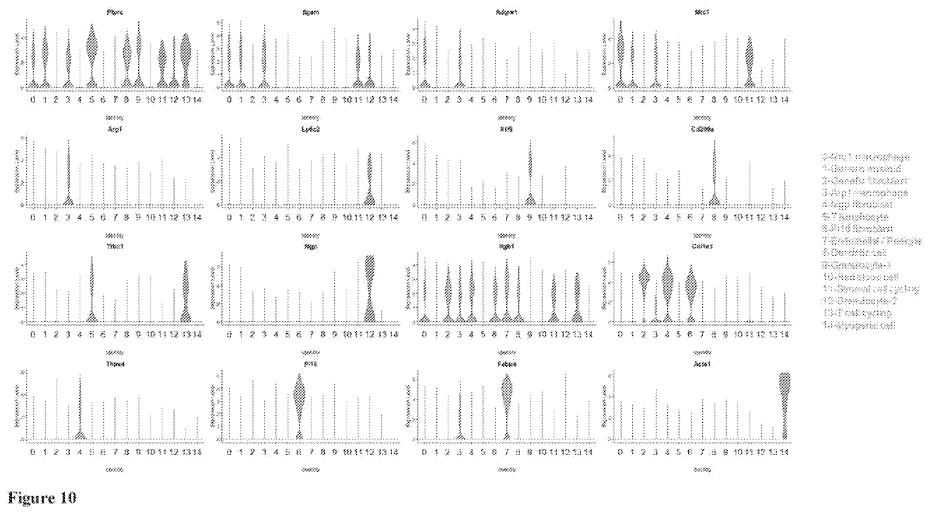

To further identify age-related signatures of injury and therapeutic response to a regenerative biomaterial therapy, we performed single cell RNA sequencing (scRNA-seq) on CD45⁺-enriched cells isolated from the muscle injuries with or without ECM treatment (FIG. 1a). We found 15 distinct cell clusters in the merged samples (FIG. 1e and FIG. 9-11) that included myeloid cells, T cells, granulocytes, fibroblasts, endothelial/pericytes, and skeletal muscle cells. We observed two different macrophage subtypes containing Mrc1 macrophage (Mrc1 Mc; Mrc1^{hi}Ccl8^{hi}) and Arg1 macrophage (Arg1 Mc; Arg1^{hi}Mmp12^{hi}) and detected multiple clusters in the fibroblast population consisting of generic fibroblast (Gen Fib; Col1a1^{hi}Col3a1^{hi}), Mgp fibroblast (Mgp Fib; Mgp^{hi}Apod^{hi}), and Pi16 fibroblast progenitor (Pi16 Fib; Pi16^{hi}Cd34^{hi}). Fibroblasts with high Mgp expression are typically defined as reticular fibroblasts²⁶⁻²⁹, and Pi16 is known to participate in regulating leukocyte infiltration and activation of the endothelial barrier³⁰. Additionally, we identified generic myeloid cell (Gen My1; Adgre1^{lo}Ccr2^{hi}Cd74^{hi}), granulocyte type 1 (Gr-1; S100a8^{hi}Il1f9^{hi}), granulocyte type 2 (Gr-2; Ngp^{hi}Camp^{hi}), T cells (Trbc2^{hi}), CD209 dendritic cell (CD209 DC; Cd209a^{hi}), a combination of endothelial cells and pericytes (Endo/Peri; Fabp^{hi}Rgs5^{hi}), and skeletal muscle cells (SMC; Acta1^{hi}Myh4^{hi}).

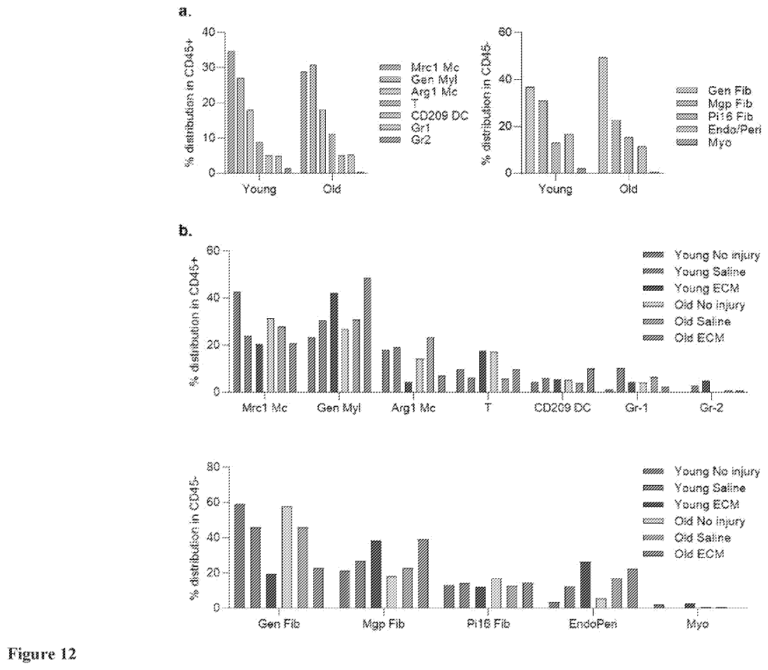

There were unique changes in cell clusters from ECM-treated muscle injuries in young or aged animals (FIG. 12). Granulocyte cluster Gr-2, enriched with Camp expression, increased after injury and ECM treatment only in young animals. Camp is associated with early transcriptional states of neutrophils^{31,32} and their cytokine and chemokine profile induces migration of eosinophils^{33,34}, supporting the increased eosinophil migration in young animals found by flow cytometry (FIG. 1c and FIG. 8). The skeletal muscle cell cluster also increased with ECM treatment only in young animals, further suggesting that ECM increased myogenic activity primarily in young mice. On the other hand, the CD209^{hi} dendritic cell cluster, whose cytokines are known to promote Th17 phenotypes in T cells³⁵⁻³⁸, increased with ECM only in aged mice. Other clusters including Gr-1, Gen My1, Mgp fib, and Endo/Peri changed with ECM treatment in a similar manner in both young and aged animals.

### Aged Animals Exhibit Impaired Immune-Stromal Interactions Required for Vascularization and Tissue Development

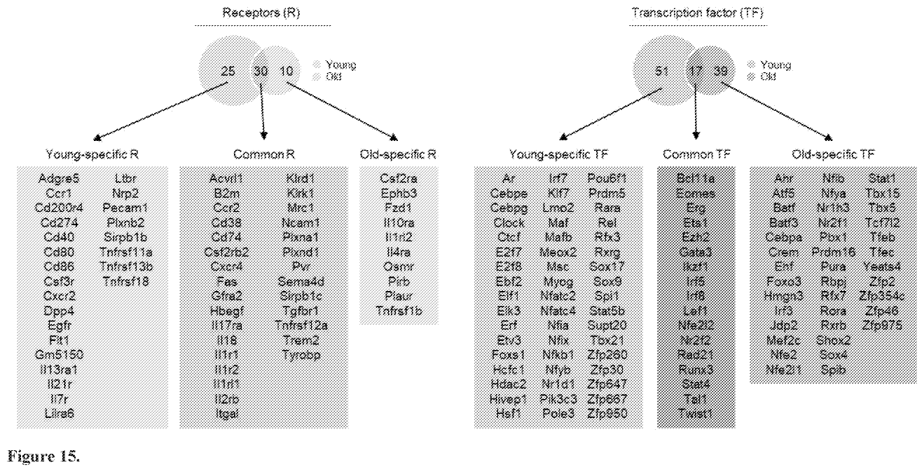

Tissue repair requires removal of debris, mobilization of stem cells, vascularization, and secretion and organization of tissue-specific extracellular matrix that is coordinated through complex immune-stromal cell interactions. To further probe the differences in the young and aged tissue environment after trauma and biomaterial application, we applied Domino to model cell-cell communication patterns using the data obtained from scRNA-seq. Domino is a computational tool that identifies condition-specific intercellular signaling dynamics based on transcription factor (TF) activation, which is surmised based on regulon expression with SCENIC gene regulatory network analysis⁴⁸, along with receptor (R) and ligand (L) expression independent of cluster⁴⁹. Domino constructs a signaling network connecting TF-R-L, which are specifically predicted to be active in the data set. TF-R connections are determined by examining correlation between R expression and TF activation scores across all cells in the data set, identifying TF-R pairings with grouped increases of expression and activation in target cell populations. R-L pairs are then determined for target receptors through the CellphoneDB2 database. In both young and old animals, a force directed diagram of the TF-R-L signaling network self-assembled into three signaling modules enriched in fibroblast, antigen processing and immune-tissue clusters (FIG. 1f, top panel). Each module indicates signaling pathways with similarly enriched activation in specific cell types. Increased module density and decreased connection across modules both indicate a group of highly correlated signaling patterns expressed in a specific cell population (both receptors and transcription factors). A complete list of the TFs and receptors corresponding to the activated TFs is provided in FIGS. 15, 16, and 32.

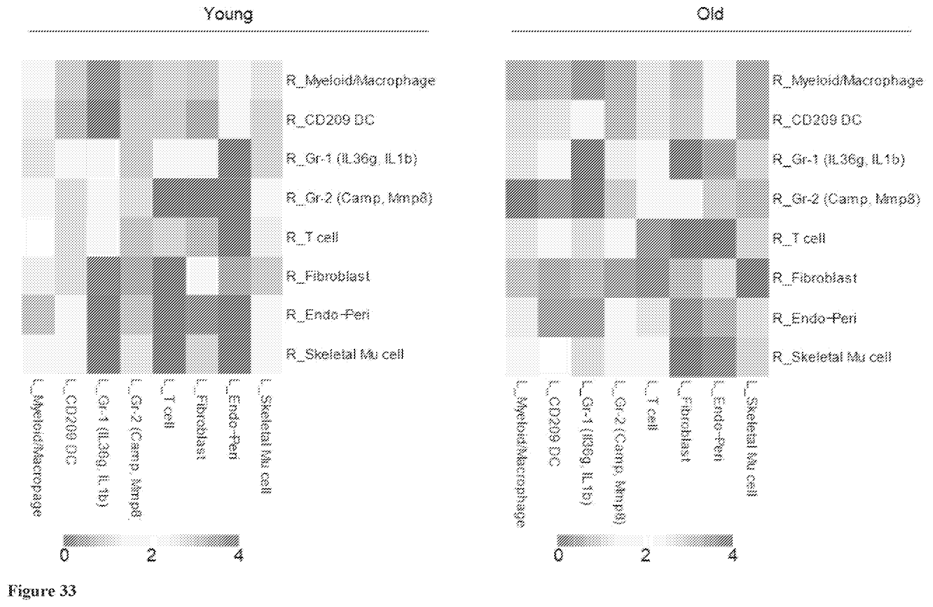

The fibroblasts in the aged tissue appeared to lose immunological properties and the narrow localization of the activated TFs suggest reduced heterogeneity (FIG. 1f, top panel). The increased connectivity between fibroblast and immune-tissue modules in the young signaling network indicates some level of plasticity in signaling in young animals, and young fibroblasts appeared to respond to the signaling patterns active in immune-tissue module. In contrast, the R and TF in the aged animals were almost completely disconnected from the other signaling modules in the aged network, indicating a less diverse role of signaling in the aged animals. None of the TF-R pairs in the aged immune-tissue module were active in aged fibroblasts. Changes in cell communication can be further identified when the single cell cluster receptor-ligand information is overlaid on the communication score (FIG. 33), and aging disruption of the immune-stromal communication can be more specifically identified (FIG. 1f, bottom panel). In particular, the stromal cluster vascular (Endo/Peri) and skeletal muscle cell (SMCs) communication with the T cells that were active in young mice during repair was severely diminished in aging mice, concordant with their reduced tissue repair⁵⁰. T cell communication with the fibroblast population was also significantly diminished in the aged mice.

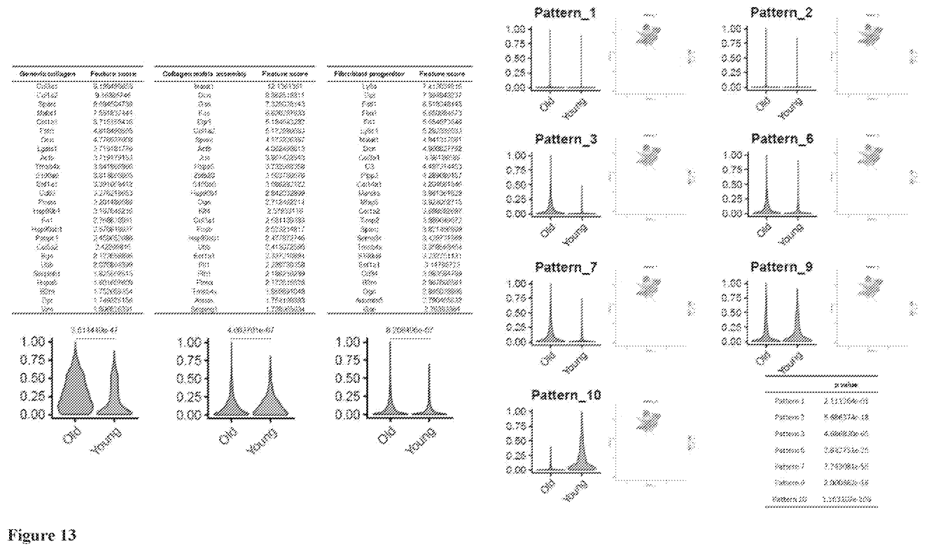

Next, we utilized a Bayesian non-negative matrix factorization (NMF) algorithm termed coordinate gene activity in pattern sets (CoGAPS) to capture additional gene sets representing cellular processes from the single cell dataset independent of changes in cellular clusters³⁹ (FIG. 1g). NMF is an alternative method to infer expression patterns that can span multiple clusters, reflective of biological processes⁴⁰, with the Bayesian framework of CoGAPS having additional sparsity constraints ideal for scRNA-seq analysis. The CoGAPS NMF analysis found gene signatures of collagen production and assembly that are dominant in the muscle injury of aged animals (FIG. 13), supporting the increased fibrosis observed histologically (FIG. 23). More specifically, gene signatures associated with collagen markers, such as Col1a1 and Col1a2, are enriched in fibroblasts from aged mice (FIG. 1g, upper panel, high expression cells in red). Furthermore, a set of genes related to collagen matrix assembly, including Dcn, Fos, and Egr1⁴¹⁻⁴³, is also more prominent in aged fibroblasts with ECM treatment showing the highest levels. When overlaid with the cell clustering dataset, the gene patterns were closely associated with specific fibroblast clusters: generic collagen genes with Gen Fib and collagen matrix assembly gene sets with Mgp Fib (FIG. 1g, upper panel circled with line).

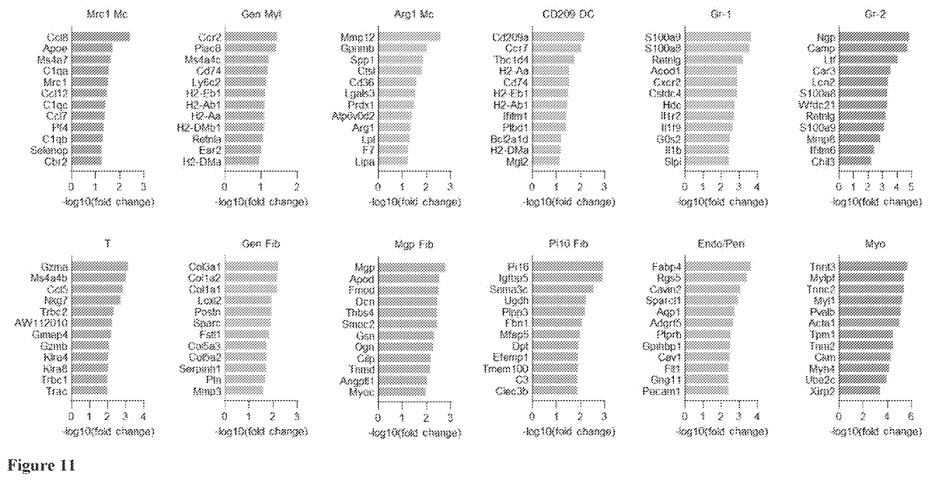

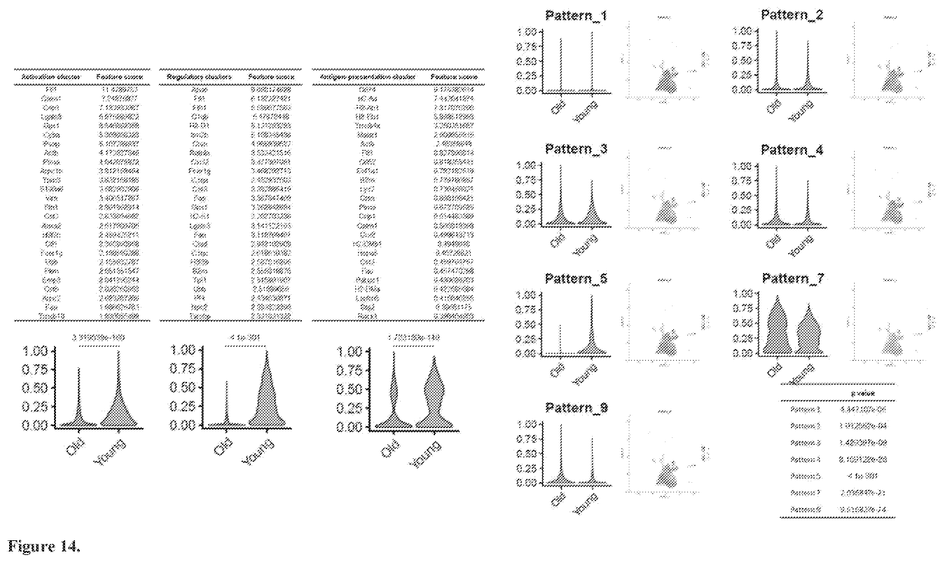

CoGAPS analysis also highlighted gene profiles that were dominant in young animals in the myeloid and macrophage cells (FIG. 1g, lower panel and FIG. 14). Genes associated with activated (Lgals3, Gpx1, Vim)^{44,45} or regulatory (Apoe, C1qb, Fcer1g)^{46,47} myeloid cells were all highly expressed in young animals compared to aged animals. While the treatment with ECM further increased the expression MHCII-associated genes (Cd74, H2-Aa, H2-Eb1) in both young and aged animals, their gene signatures were much more distinct within the young. When overlaid with cell clusters, we found an MHCII-associated gene pattern predominant in the Gen My1 cluster (FIG. 11), which increased with ECM treatment (FIG. 12). The majority of additional gene patterns in myeloid and macrophage cells were also dominant in the young mice (FIG. 14). This suggests that even though some cell clusters increased with ECM treatment in a similar manner between young and aged mice, they may have different gene signatures that impact functional outcomes. Furthermore, the loss of myeloid function in older animals may contribute to the impaired regeneration response to tissue injury and ECM biomaterials.

Altogether, the flow cytometry and single cell analysis demonstrate that key immune populations involved in muscle repair and a regenerative therapeutic response, such as eosinophils and CD4 T cells, decrease with aging. Furthermore, aging increases proinflammatory cells such as CD8 T cells and increases fibrosis signatures in response to regenerative treatments while at the same time decreasing immune activity features in myeloid and macrophages cells relevant for tissue repair including antigen presentation and mobilization.

### Aged Animals Exhibit Reduced Cell-Cell Communication Networks and Impaired Fibroblast Interactions

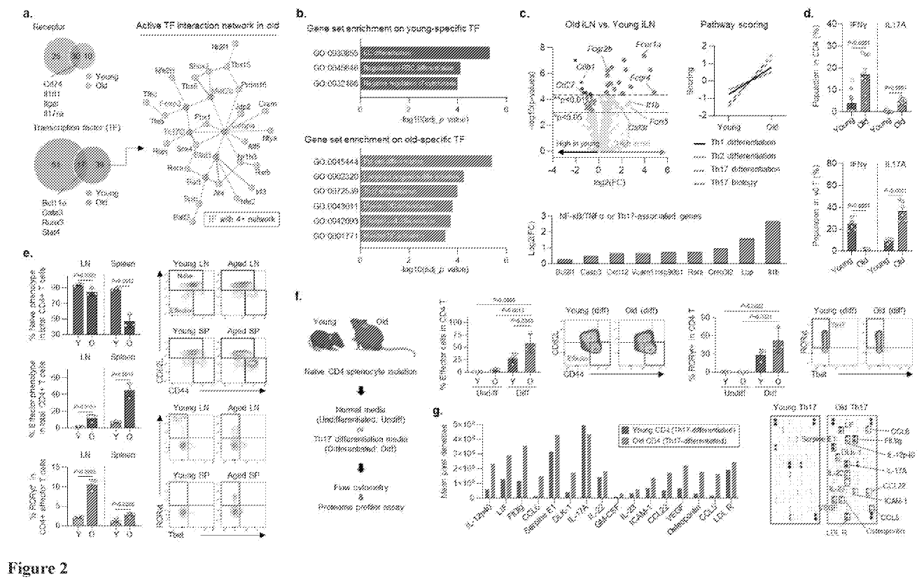

To further probe the differences in the young and aged tissue environment after trauma and biomaterial application, we applied Domino analysis to model cell-cell communication patterns using the data obtained from scRNA-seq (FIG. 2). Domino is a computational tool that identifies condition-specific intercellular signaling dynamics based on transcription factor (TF) activation, which is surmised based on regulon expression with SCENIC gene regulatory network analysis⁴⁸, along with receptor (R) and ligand (L) expression independent of cluster identification⁴⁹. Domino constructs a signaling network connecting TF-R-L which are specifically predicted to be active in the data set. In both young and old animals, a force directed diagram of the TF-R-L signaling network self-assembled into three gene modules enriched in fibroblast, antigen processing and immune-tissue clusters (FIG. 34). Each module indicates signaling pathways with similarly enriched activation in specific cell types, and connections between the modules indicate signaling patterns which are active in more than one cell type. The fibroblast module in aged mice demonstrated a limited connection with other modules, suggesting impaired communication between immune and stromal populations. The antigen-processing module, represented by macrophages, myeloid cells, dendritic cells and T cells, interacts with both the fibroblast and immune-tissue module in young mice muscle. In aged animals, however, the fibroblast module reduced to only one connection with antigen processing and no interactions with the immune module, consistent with the CoGAPS results that showed limited immune activity features in myeloid and macrophage cells of aged mice.

Lack of inter-modular signaling with aging became more distinguished when inter-cluster dynamics are analyzed (FIG. 34, bottom panel). Clusters such as S100a8hiI11f9hi Gr-1, Endo/Peri, and T cells demonstrated active communication networks with other cell populations in young mice, however, their inter-cluster crosstalk diminished in old mice. While there was still some communication among cell clusters in aged animals, their signaling was much weaker compared to young animals. In particular, the lack of communication with the Endo/Peri cluster is likely relevant to muscle tissue regeneration because muscle stem cells and satellite cells are known to recruit endothelial cells to secrete growth factors such as IGF-1, HGF, bFGF, and VEGF to promote satellite cell growth, thereby affecting the degree of capillarization of the myofibers, angiogenesis and ultimately myogenesis⁵⁰.

### Unique Transcription Factor Activation Identifies a Type 3 Immune Profile in Aging

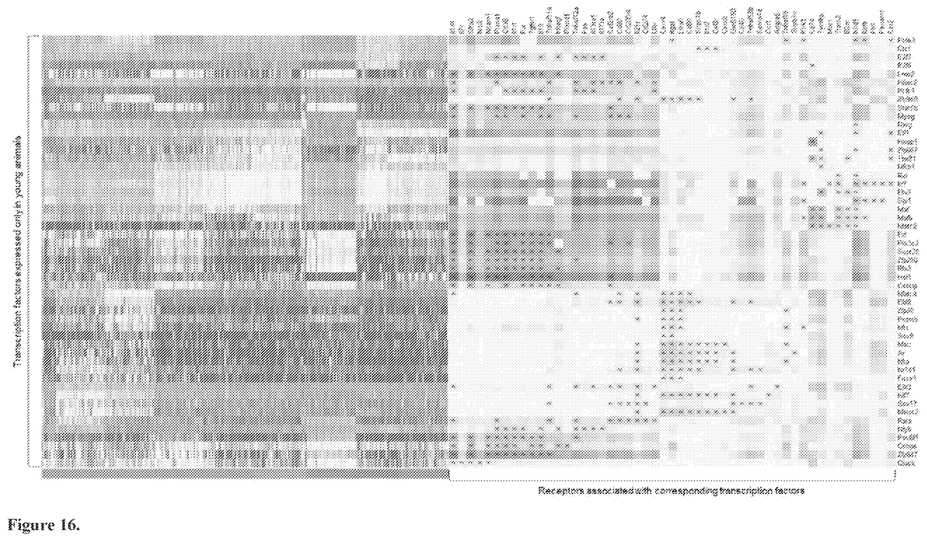

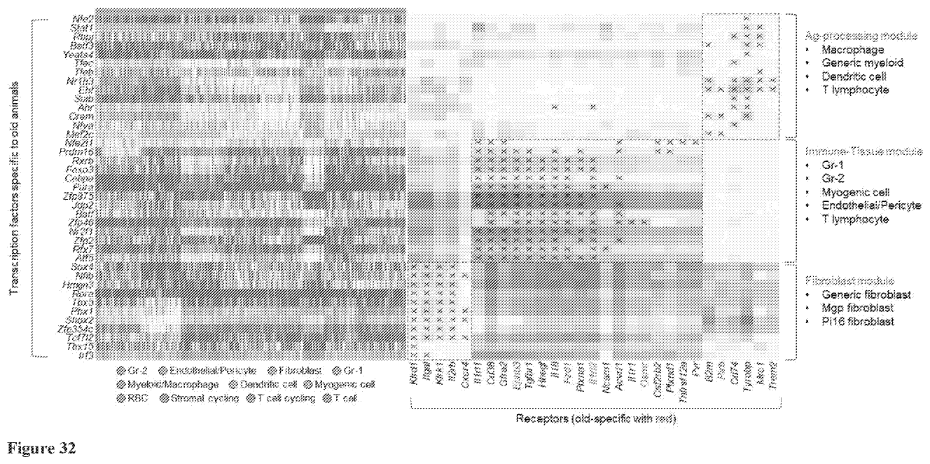

To identify signaling components that may be responsible for reduced cell signaling and impaired wound healing with age from the single cell and Domino analysis, we compared the transcription factors and receptors active in the young and old networks (FIG. 2a, FIG. 15, FIG. 16 and FIG. 32). We identified several receptors common in both young and old signaling networks that correlated with activation of different TFs depending on age (FIG. 16 and FIG. 32). These TFs are connected to IL4 and IL17 immune cytokine expression which negatively regulate each other to reinforce the differentiation pathways that were disrupted in the aging tissue. For example, we found transforming growth factor beta receptor 1 (Tgfbr1) in the immune-tissue module of young and aged animals correlated with Batf activation only in aged mice. Batf is one of the activator protein-1 (AP-1) proteins that controls Th17 differentiation⁵², supporting the type 3 immune profile with aging. Additionally, a signal transduction adaptor Tyrobp, which was expressed in antigen processing modules in both age groups, correlated with Batf3 activation, which regulates Th2 cell function while inhibiting the differentiation of regulatory T cells^{58,59}, only in old mice.

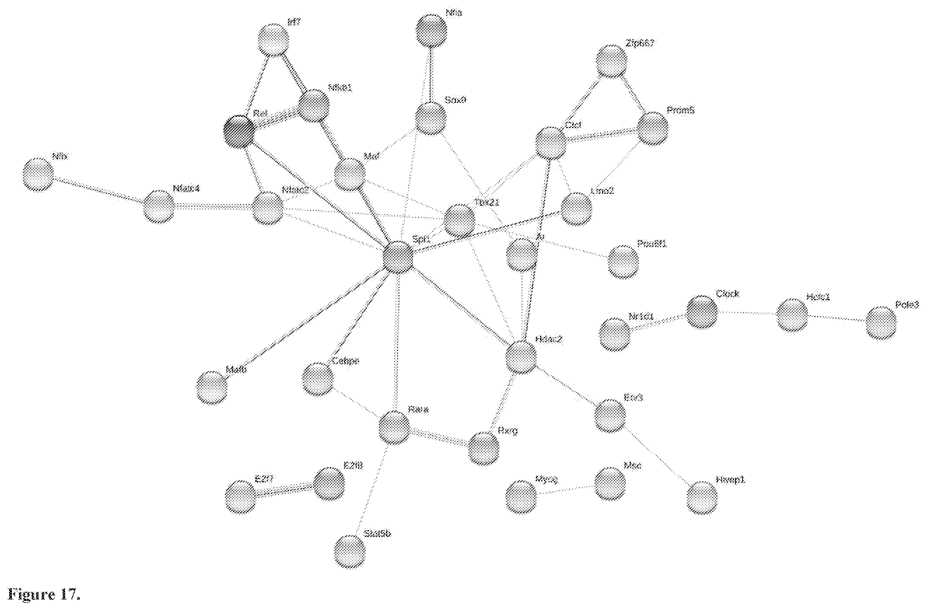

We then used String network to examine protein-protein interactions between the age-specific transcription factors (FIG. 2a right panel and FIG. 17). The resulting network highlighted the aged animals-specific transcription factors, Foxo3, Mej2c, Tcf7l2, Cebpa, Stat1, and Batf as integral to the unique aging immune transcriptional network. To understand the biological significance of the age-specific TFs, we then performed gene set enrichment (GSE) analysis using Enrichr^{64,65}, and enriched gene ontology (GO) terms to classify the biological processes in which they function (FIG. 2b). The young-specific TFs were associated with pathways involved with regulation of red blood cell differentiation or cytokinesis, and more importantly, endothelial cell differentiation. On the other hand, aged animal-specific TFs were enriched for fat cell differentiation, Th17 differentiation and myeloid DC differentiation pathways. Signaling associated with Th17 differentiation is known to negatively regulate eosinophil recruitment and IL4 expression^{54,66,67}, suggesting that this immunological skewing in the TFs with aging may be responsible for the impaired Th2 response to the regenerative ECM biomaterial in the old animals.

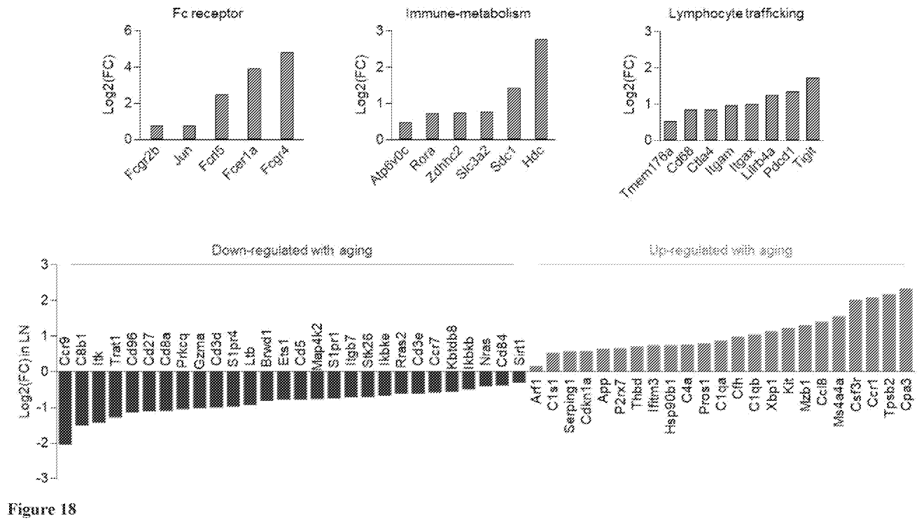

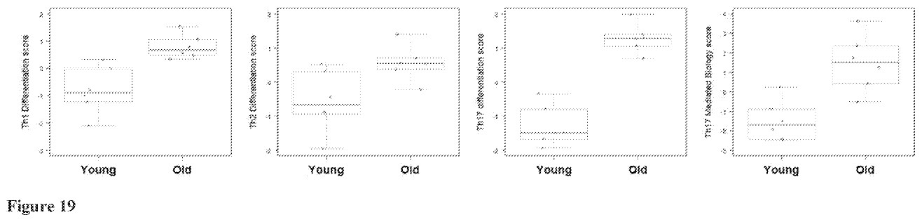

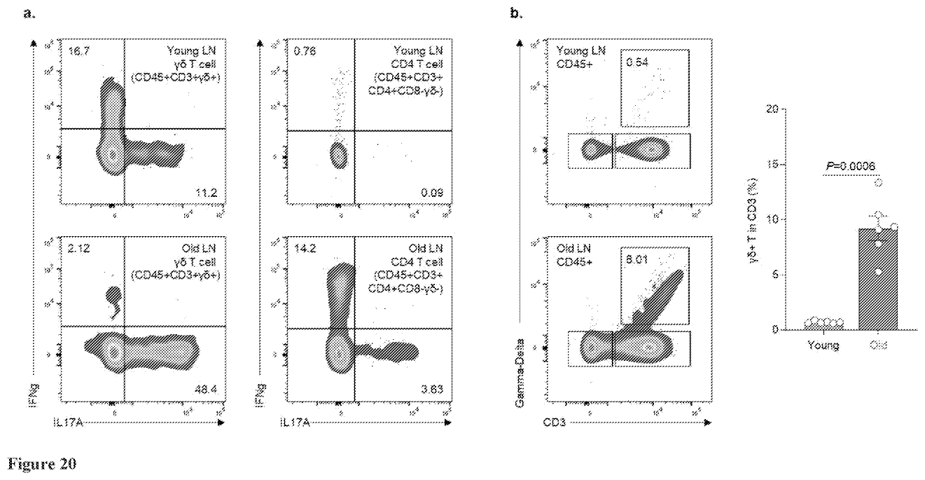

To validate the computationally predicted type 3 immunological skewing in aged animals, we analyzed the baseline proximal inguinal lymph node (iLN) properties before injury. We first compared the gene expression profile of iLNs from the naïve (no injury) young and aged mice using Nanostring analysis (FIG. 2c and FIGS. 18-19) and flow cytometry (FIG. 2d and FIG. 20). Aged lymph nodes expressed lower levels of general T cell markers such as Cd3e and Cd3d, consistent with the known decrease in T cell numbers with aging. However, the expression of NF-κb/TNFα-, Fc receptor- or Th17-associated gene sets, all of which are potent inducers of Th17-mediated inflammation⁶⁸, increased in the aged lymph nodes (FIG. 2c and FIG. 18). Pathway scoring further suggested that aging promoted T cell activation and skewing in Th17 differentiation or Th17-biology related gene expression (FIG. 2c and FIG. 19). Flow cytometric analysis further supported a type 3-skewed immune environment in the aging lymph node with a significantly higher proportion of IL17-producing CD4 and γδ T cells present in the aged iLN compared to the young (FIG. 2d and FIG. 20). While both the Th1 (IFNγ⁺ CD4) and Th17 (IL17a⁺ CD4) cells increased with aging, the γδ T cells switched phenotype from type 1 (IFNγ⁺) to type 3 (IL17⁺).

### Aging CD4 T Cells Demonstrate a Unique Th17 Immune Phenotype

To further evaluate the age-associated type 3 immune signatures, we evaluated whether aging CD4 T cells have a higher propensity for Th17 differentiation compared to young CD4 T cells (FIG. 2e-g). As expected, CD4 T cells isolated from the lymph nodes and spleen of aged animals exhibited fewer naïve (CD44−CD62L−) but significantly higher percentages of effector (CD44+CD62L−) CD4 T cells compared to the young with the greatest difference found in cells isolated from the spleen (FIG. 2e). The effector T cells had notably higher percentages of RORγt expression, a lineage defining transcription factor of Th17 cells, particularly in cells isolated from the lymph node. To determine if the increased numbers of Th17 cells (FIG. 2e) was due to an increased propensity for differentiation, we isolated naïve CD4 T cells from the spleen of young and old animals, and cultured in Th17 skewing conditions in vitro (FIG. 2f). Naïve CD4 splenocytes from aged animals demonstrated significantly more effector cells and RORγt⁺ Th17 cells when cultured in skewing conditions compared to young animals (FIG. 2f, right panel). In addition to increased skewing into RORγt⁺ Th17 cells during in vitro culture, T cells from aged animals had a different secretome after skewing. Proteome analysis on the cells differentiated from naïve aging CD4 T cells showed significant upregulation in inflammatory cytokines, including IL12p40, a subunit for IL-23 that is required for Th17 differentiation, IL-6 family leukemia inhibitory factor (LIF), CCL5, CCL6, CCL22 and many others (FIG. 2g). This data suggests that naïve CD4 T cells from aging environment differentiate into a unique Th17 phenotype, and aging Th17 cells demonstrate a different secretory profile compared to young Th17 cells including more VEGF and CCL5, both of which play a direct role in angiogenesis.

### Aging Induces a Local and Systemic Type 3 Immune Response to Injury and Biomaterial Therapy

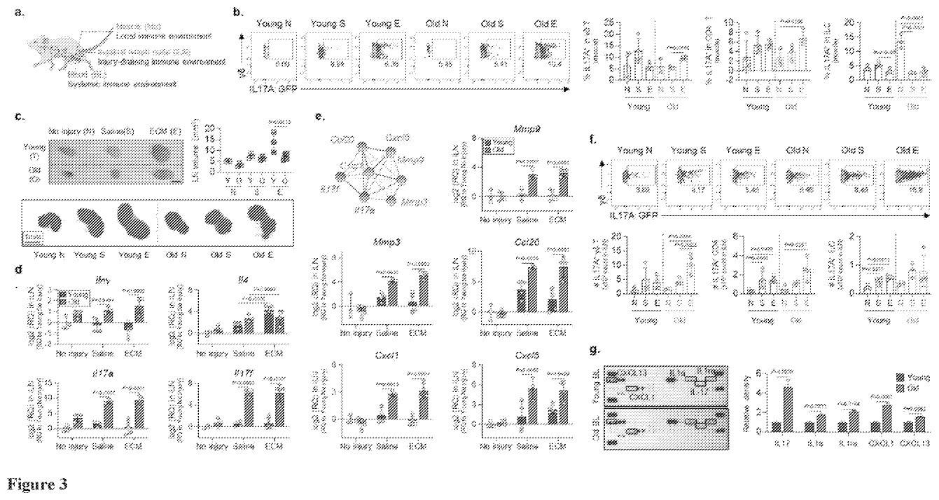

We analyzed more in depth the source of immune dysfunction after injury and ECM treatment in old animals (FIG. 3). Cytokine gene expression and protein profiles in the muscle tissue differed with age (FIG. 3b, 36). 114 gene expression, critical for muscle repair, increased in muscle tissue only in young animals after injury with further significant increases after ECM treatment (FIG. 3b). In aged animals, injury did not increase 114 expression in muscle, and its level of gene expression after ECM treatment was significantly lower than the young animals with ECM. Instead, ECM treatment in aged mice increased the expression of Il17f (Il17a gene expression was not detected in the muscle tissue of young or aged mice). Other inflammatory genes, including Ifnγ, Il23a, Il6, Il1b, along with Cdkn2a and S100a4, all increased similarly in both young and old mice with injury or ECM treatment (FIG. 21).

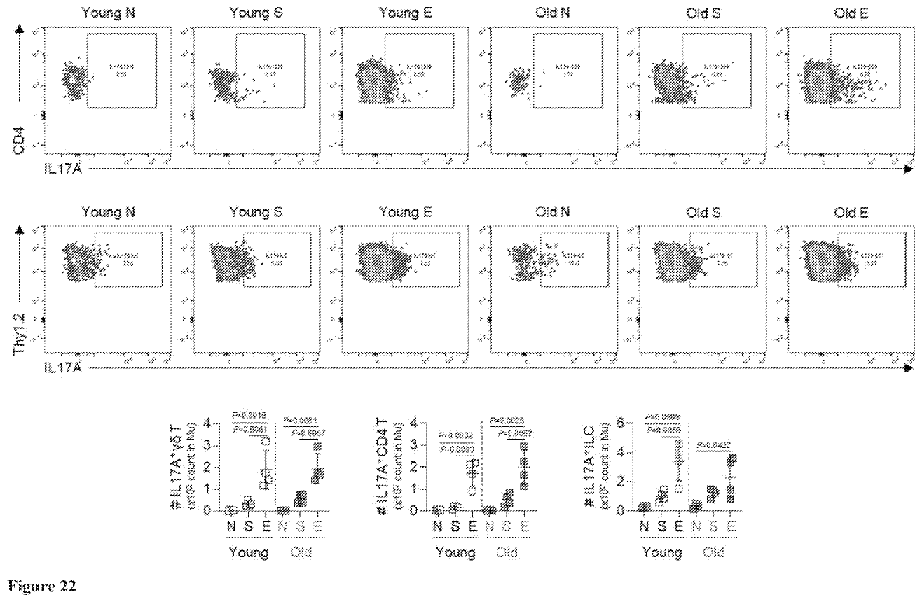

As the aged animals have higher baseline IL17 expression that increases significantly after injury and ECM treatment, we next analyzed in detail the source of IL17 (FIG. 3). Using young and aged IL17A-IRES-GFP-KI (IL17A-GFP) mice, we found γδ T cells, CD4 T cells, and innate lymphoid cells (ILC) all expressed IL17A after injury and increased in number after ECM treatment in both young and aged mice (FIG. 22). The percentage of IL17A⁺γδ T cell, however, only significantly increased in aged animals with ECM treatment compared to injury alone and the percentage of IL17A⁺ CD4 T cells increased only in aged animals with ECM treatment compared to no surgery (FIG. 3b). The percentage of ILCs expressing IL17A was similar among all groups except for naïve aged muscle tissue where there were significantly higher baseline levels, albeit in small cell numbers.

To determine the functional impact of the different immune environments in the young and aged animals on the ECM response and tissue repair, we evaluated the tissue histology 1 week after injury and treatment (FIG. 1d and FIG. 23). There was significant cell infiltration after injury in both young and aged mice, and ECM treatment further increased cellular infiltration and the presence of collagen as visualized by Masson's Trichrome staining. Consistent with the GSE analysis that demonstrated age-associated TFs were enriched for fat cell differentiation (FIG. 2b), there was excessive adipose tissue in the muscle of aged animals compared to the younger counterparts, which increased further with ECM treatment.

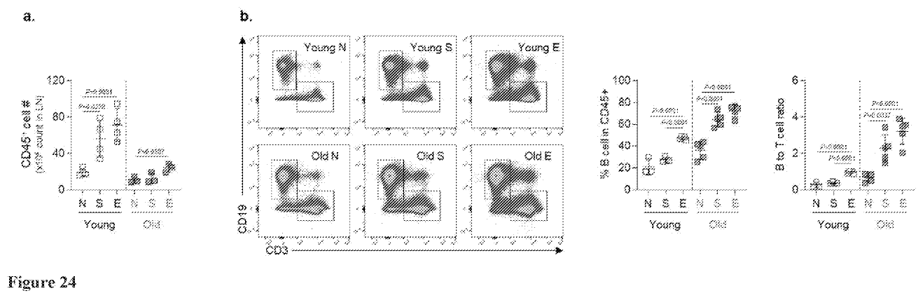

We then assessed the regional and systemic response to injury and ECM treatment in the lymph nodes and blood from young and aged mice (FIG. 3c-g and FIG. 24-26). First, we observed visible differences in the size of the draining (inguinal) LNs with the young LNs appearing much larger before and after injury and increasing further in size with ECM treatment compared to the aged animals (FIG. 3c). Quantification of iLN volume using caliper measurements confirmed a significant increase in size in the young animals with ECM treatment. The LN size correlated with total cell numbers in iLN with injury and ECM treatment significantly increasing the number of CD45⁺ cells in young animals, while only ECM treatment significant increased cell numbers in the aged animals albeit to a level significantly lower than young animals (FIG. 24). In contrast to the lymph node, muscle tissue did not demonstrate significant differences in total CD45⁺ cell number between young and aged animals after ECM treatment (FIG. 7), suggesting that the lymph node highlights the age-associated immune and regenerative dysfunction that may be responsible for poor muscle regeneration with age.

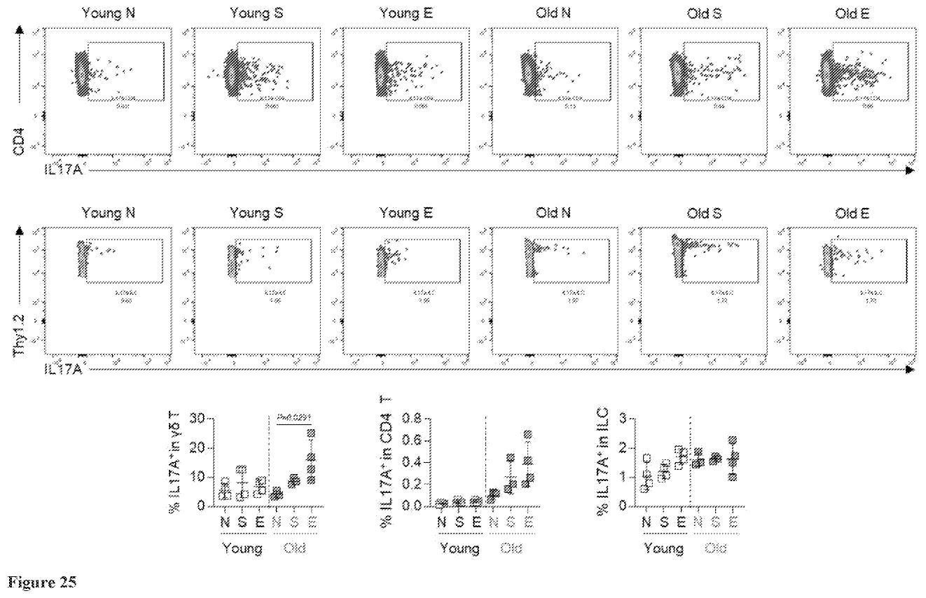

Gene expression for canonical cytokines in the lymph node correlated with the immune profiles found in the muscle tissue. ECM treatment significantly increased 114 expression in young iLNs only (FIG. 3d) and there were minimal changes in Ifnγ expression. Strikingly, both Il17a and Il17f expression significantly increased only in aged animals after injury and ECM treatment. We further analyzed the expression of genes that relate to Th17 skewing, as defined by String network analysis; Mmp3, Mmp9, Cxcl1, Ccl20, and Cxcl5⁶⁹. Expression of these genes occurred at low levels in all conditions in naïve young and aged animals but significantly increased only after injury and ECM treatment in aged mice (FIG. 3e), suggesting that injury further triggered a type 3 immune response beyond the baseline in an aging tissue environment. Multiparametric flow cytometry analysis of the cells from the iLN further confirmed the trend with increasing numbers and percentages of IL17A⁺γδ and CD4 T cells after ECM treatment only in aged mice (FIG. 3f and FIG. 25). In addition, B cells also exhibited age-specific changes after injury and ECM treatment (FIG. 24). Similar to the increased number of B cells in aging muscle tissue (FIG. 7), iLN from old animals had significantly higher percentages of B cells after injury and treatment, further skewing T to B cell ratio with age.

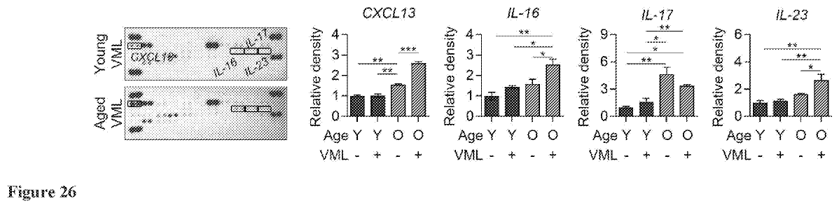

To further assess the systemic immune changes with aging, we analyzed cytokines in serum using proteome profiler (FIG. 3g and FIG. 26). In addition to increased IL17 in the serum of aged mice, there was also increased level of the B cell chemoattractant factor CXCL13, suggesting that the increased B cell signatures in aged muscle and lymph nodes also extended systemically. CXCL13 also significantly increased in blood after injury in old mice (FIG. 26), further supporting that injury promotes a B cell response solely in old animals where the overall number of T cells and naïve T cells is significantly reduced. Additionally, IL1A, a potent inducer of IL17 from T cells⁷⁰, and CXCL1, one of IL17-induced chemokines, also increased in serum from the aged mice. IL23, a protein that can expand Th17 cells⁷¹, increased in response to injury only in old mice. Collectively, these data show that injury and ECM treatment in aged animals triggered a local and systemic type 3-mediated immune response that may be responsible for the decreased regenerative and therapeutic response with aging.

Inhibition of IL17 Rejuvenates Type 2 Response after Muscle Injury in Aged Animals

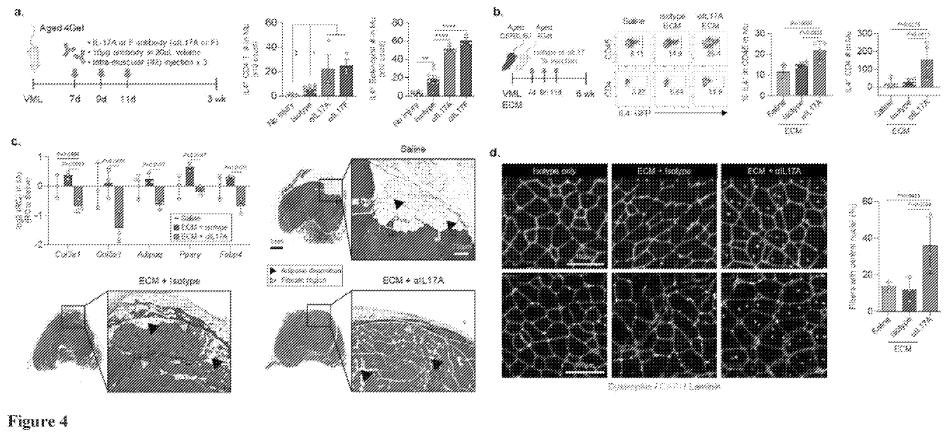

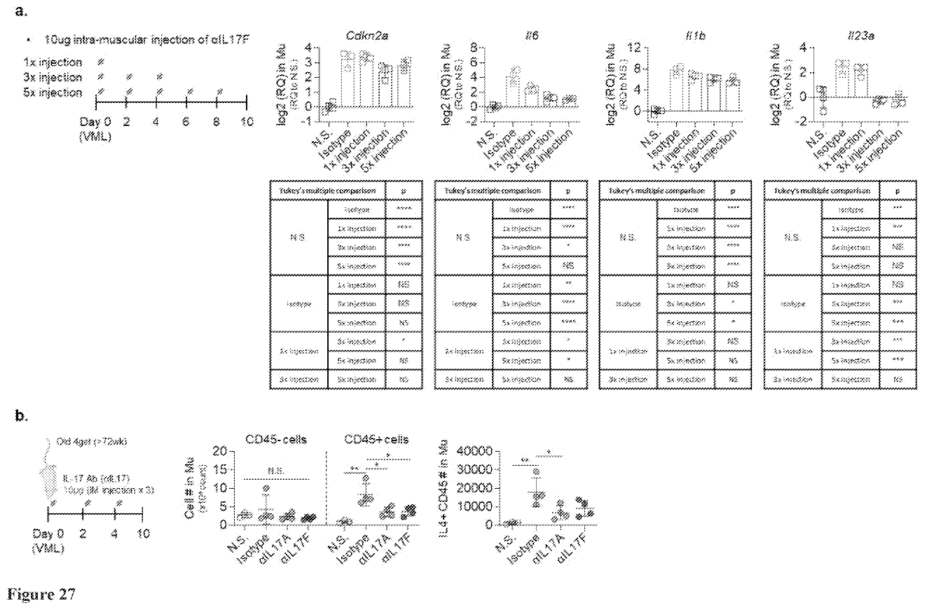

Since IL17 is associated with fibrosis^{72,73} and negatively regulates IL4 that is needed for tissue repair, we investigated if IL17 neutralizing antibodies (αIL17) could restore IL4 expression and tissue repair that is lost with aging. We first evaluated the dose and timing for delivery (FIG. 27). A minimum of three injections was required to reduce inflammatory markers in the tissue after injury in aged mice. However, initiating αIL17 injections at the time of injury stunted infiltration of immune (CD45⁺) cells including IL4⁺ cells that are critical for tissue repair, suggesting the importance of acute IL17 during wound healing to attract the immune effectors and initiate tissue regeneration (FIG. 27b). We then tested initiation of αIL17 injections one week after injury to allow immune infiltration before blocking (FIG. 4a). Using aged Il4^{tm1Lky} mice (4Get), which have a fluorescent reporter for IL4 expression, we found that both αIL17A and αIL17F treatment one week after injury significantly increased the number of IL4⁺ eosinophils and CD4 T cells three weeks after injury.

### Combination Therapy of Pro-Regenerative ECM and αIL17 Rejuvenates Muscle Repair in Aged Mice

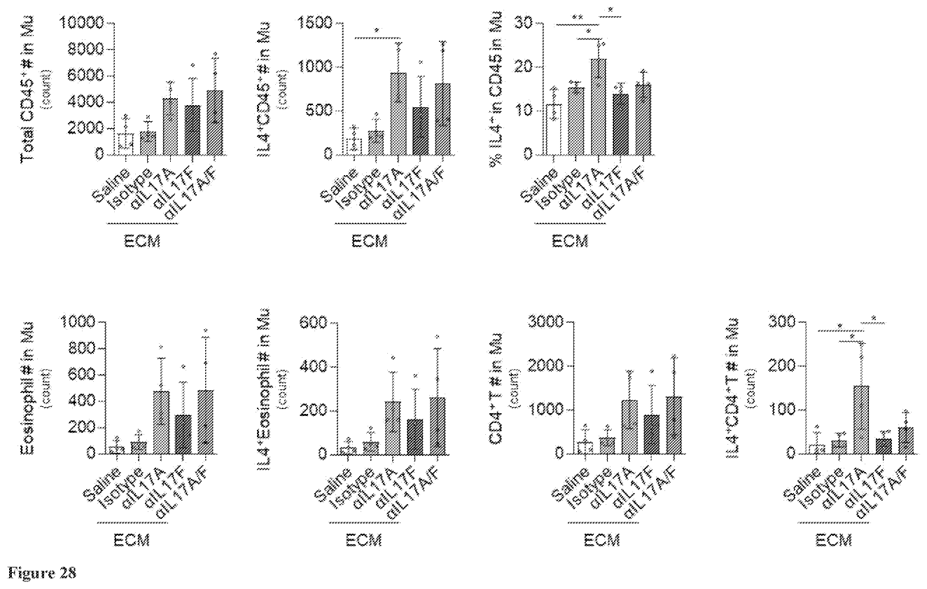

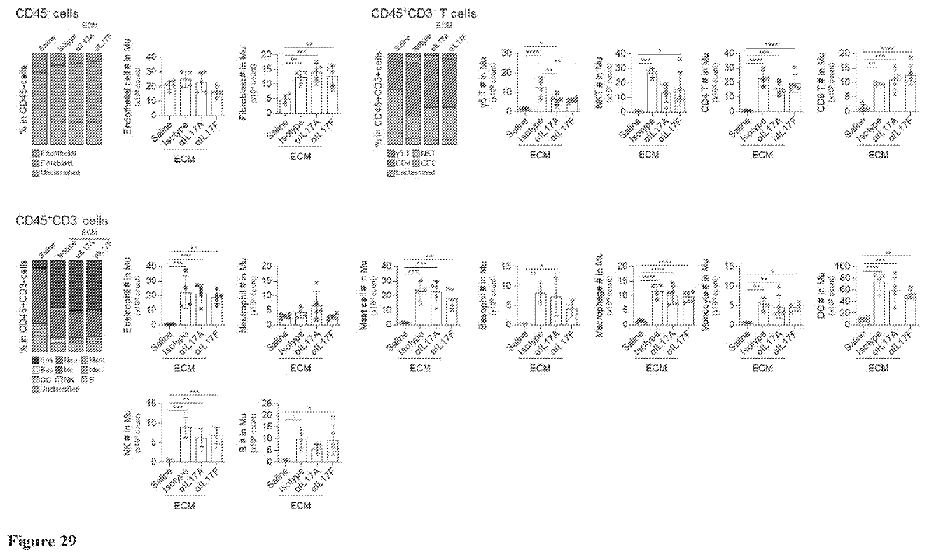

Since αIL17 treatment alone restored IL4 expression after injury in aged animals, we tested a combination therapy approach with pro-regenerative ECM and αIL17 (FIG. 4b). Muscle wounds in aged animals received ECM treatment at the time of surgery and local αIL17 one week after injury. Six weeks after injury in aged 4Get mice, the antibody treatment after ECM implantation increased CD45⁺ cell numbers (FIG. 28), however, only the αIL17A treated animals had a significantly higher number and percentage of IL4⁺CD45⁺ immune cells, and number of IL4⁺CD4⁺ T cells compared to the αIL17F or ECM only groups, albeit in small numbers at this later time point. Three weeks after combination therapy, we observed a significant decrease in γδ T cell numbers, a major source of IL17, in mice treated with αIL17A or αIL17F using multi-spectral flow cytometry (FIG. 29).

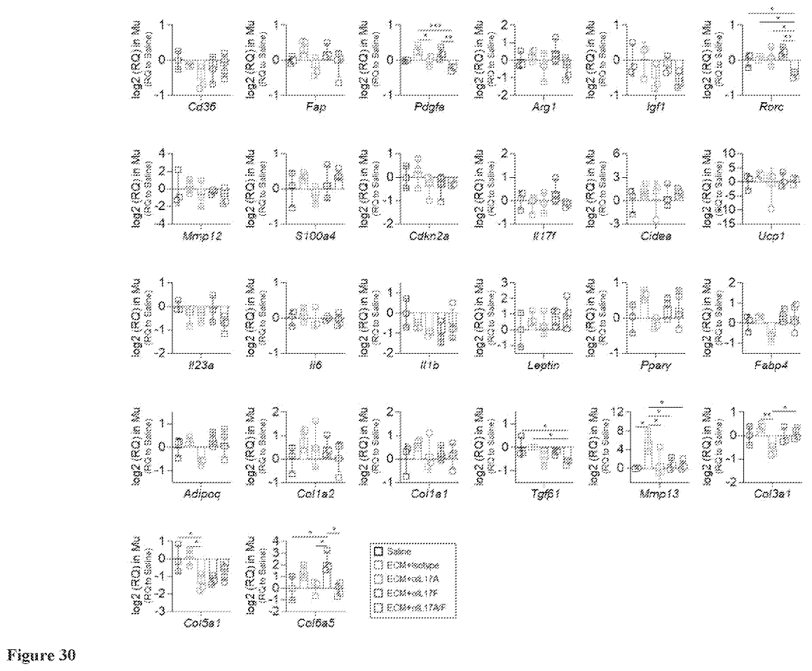

We then explored the therapeutic response to ECM-αIL17 combination in the muscle wound and tissue repair. Six weeks after injury, muscle tissue from aged C57BL/6J mice treated with ECM and αIL17A expressed significantly lower levels of numerous fibrosis- or adipose-associated genes that CoGAPS analysis identified as increasing in aged animals (FIG. 4c, top left panel). Specifically, collagen type III a1 (Co3a1) and collagen type V a1 (Col5a1) decreased significantly only with ECM-αIL17A combination treatment. Additionally, adipose-associated genes, such as Pparγ, Fabp4, and Adipoq all dramatically decreased with αIL17A treatment. Other fibrosis-related genes, such as Fap and Pdgfa⁷⁴, decreased with αIL17A or αIL17A/αIL17F (FIG. 30). Expression of the inflammatory gene Mmp13 significantly decreased with any combination of αIL17 treatments, however, RAR-related orphan receptor type c (Rorc), which is critical in the differentiation of Th17⁷⁵, was only downregulated with a combination of αIL17A/αIL17F treatment. Differences in gene expression profile between the αIL17A and αIL17F-treated groups suggest that there may be a distinction between IL17a and IL17f signaling pathways.

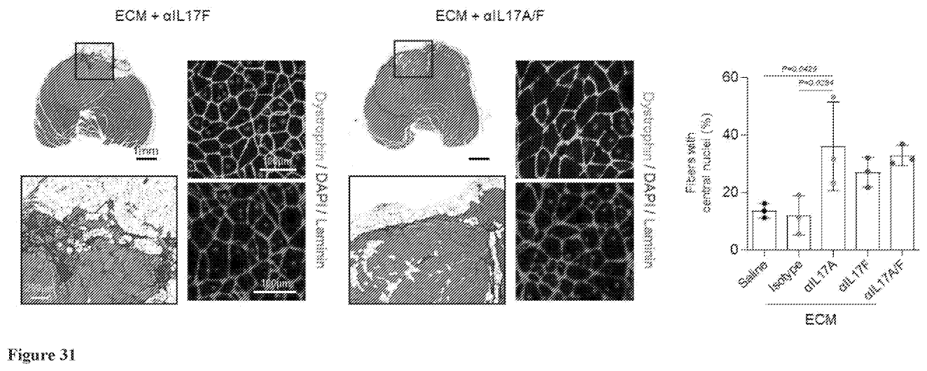

Histological evaluation of the muscle defect in aged animals treated with combination therapy supported the immunological and gene expression results with increased repair and reduced fibrosis depending on the form of IL17 neutralization (FIG. 4c and FIG. 31). Aged mice treated with ECM alone demonstrated collagen deposition and fibrosis that decreased with αIL17A or combination αIL17A/αIL17F treatment as visualized by Masson's trichrome. Adipose deposition in the repaired tissue also decreased with ECM-αIL17A combination treatment as visualized in the histology, supporting the gene expression results. Additionally, a combination treatment induced nuclear repositioning from the periphery to the center of muscle cells (FIG. 4d), a characteristic of repairing muscle tissue that did not occur in animals treated with injury or ECM alone. Nuclear positioning is critical in muscle fiber function, and it was shown that myofibers that are regenerated several weeks after muscle damage can be characterized by centrally localized nuclei^{76,77}. Quantification of muscle cells with centrally-located nuclei demonstrated that ECM-αIL17A combination treatment significantly increased the percentage of regenerating muscle fibers compared to the injury or ECM alone groups (FIG. 4d, right panel). Altogether, we demonstrate that aging significantly alters the immune and stromal response to muscle injury and therapeutic biomaterials in the local tissue, regional lymph nodes and systemically in blood. These altered responses, characterized by increased IL17, reduced IL4 and excess fibrosis and adipogenesis, can be mitigated with a combination therapy of a pro-regenerative ECM material and an IL17 antibody to promote the repair capacity in aging tissue.

### Analysis

In this work, we uncovered age-related changes associated with type 3 (IL17) immunity that are present in secondary lymphoid organs, that is further exacerbated after muscle injury and treatment with a regenerative ECM biomaterial. Repaired tissue in the aged animals is characterized by excessive fibrosis and adipose tissue with treatment. Single cell analysis revealed excessive collagen activity and abnormalities in myeloid and antigen presentation in the aged animals. Cell communication highlighted diminished immune-stromal cell interactions with aging, particularly between aged T cells, which had an altered secretome, and vascular-related clusters (endo/peri) and muscle cells. Combination therapy of the ECM scaffold with an IL17 neutralizing antibody in the aged animals restored, in part, the pro-regenerative immune response and tissue repair while reducing fibrosis and excess adipose.

Tissue injury mobilizes the immune system and uncovers new age-associated dysfunctions that may not be otherwise apparent. Aging is associated with numerous chronic diseases and increased incidence of cancer⁷⁸. Healthy aging though, even without overt disease, results in longer recovery times from tissue injury. Changes in cellular composition with aging may be in part responsible for reduced healing capacity including decreased endogenous stem cell numbers and activity, in addition to reduced fibroblast heterogeneity⁸⁹. However, the pivotal role of the immune system in the response to tissue injury and directing tissue repair is critical to consider as there are many age-related changes in the immune system. Even the epigenetic changes that have been implicated in age-associated repair dysfunction⁷⁹ may extend to the aging immune response to tissue damage as we observed a different secretome of aged Th17 skewed cells cultured in similar conditions to young T cells that is likely due to epigenetic changes. As regenerative medicine strategies are moving to target the immune system, understanding these age-associated immune changes will be critical to develop regenerative immunotherapies that are relevant to the older patient populations that are more likely to suffer from delayed or inadequate tissue repair. Finally, as biological age does not always correlate with chronological age, relevant diagnostics and personalized therapeutic approaches may be needed.

While multiple regenerative medicine therapies are available, we chose ECM biomaterials to evaluate in an aging environment because of their clinical use¹⁶.

ECM biomaterials derived from allograft and porcine sources are approved for wound healing and reconstructive surgery applications, orthopedic, and ophthalmologic indications^{16,81,82}. ECM materials contain a complex mixture of proteins, proteoglycans, and even matrix-bound vesicles that likely all contribute to damage signals and other as yet determined factors that mobilize multiple immune and stromal cell types to promote tissue repair.

Aged animals exhibited a baseline inflammatory state with more CD8⁺ T cells and Th17 cells, the latter being most predominant in the lymph nodes. In the muscle tissue, however, IL17 expression was only observed after injury and ECM treatment, which induced the most significant increases in IL17. While Il17a and f were observed in the lymph node, only Il17f gene expression was found in the muscle tissue. Injury in the older animals uncovered many age-related signatures associated with IL17 and its signaling, and this was further exacerbated with ECM implantation. The cytokine IL17 is a component of the host defense against extracellular pathogens^{55,66}, but is also associated with fibrosis and fibrotic disease^{72,73}, suggesting a common mechanism of “walling off” uncontrolled pathogens and maintaining barrier surfaces and microbiome balance. While IL17 is important for the recruitment of effector immune cells for wound repair and host defense, its chronic state with aging can further induce carcinogenesis, fibrosis, and inappropriate immune responses. Age-associated commensal dysbiosis may contribute to the excess IL17 in addition to senescence-induced immunomodulation that promotes IL17⁸⁸. As mice are reared in a controlled lab environment, the increased aged-associated IL17 related to gut dysbiosis may be even greater and more variable in people that have more diverse environment exposure, diet, and etc.

Cell communication analysis by Domino uncovered active immune-stromal module interactions in young animals that were impaired and limited in an aging environment. Young mice demonstrated immune-stromal communication associated with vascular development and muscle cell activity, both of which are well recognized for their roles in tissue repair. Interestingly, the aged Th17-skewed cells secreted more VEGF in vitro compared to the young T cells, suggesting a possible epigenetic memory associated with vascular insufficiency. In addition to VEGF, the aged Th17 cells also secreted increased levels of LIF, a component of the Stat3 and Wnt signaling pathway involved in vascular development. Vascular insufficiency and impaired VEGF signaling is a hallmark of aging, particularly in the microvasculature which is a necessary component of tissue repair regeneration⁸⁹.

In summary, the immune system represents a new therapeutic target for regenerative medicine. However, the complexity of the immune system in people and variability related to intrinsic genetic, sex differences, exposure history and environmental factors that only increases with age must be considered in therapeutic design. Combination therapies, a standard approach in cancer treatment, should be extended to regenerative medicine where complex interactions between the immune system, stem cells, and the vascular system contribute to repair outcomes.

### REFERENCES

1 Kirkwood, T. B. Understanding the odd science of aging. Cell 120, 437-447, doi:10.1016/j.cell.2005.01.027 (2005).
2 Pisco, A. O. et al. A single cell transcriptomic atlas characterizes aging tissues in the mouse. BioRxiv, 661728 (2019).
3 van Deursen, J. M. The role of senescent cells in ageing. Nature 509, 439-446, doi:10.1038/nature13193 (2014).
4 Du Nouy, P. L. Cicatrization of wounds: III. The relation between the age of the patient, the area of the wound, and the index of cicatrization. J Exp Med 24, 461-470 (1916).
5 Gosain, A. & DiPietro, L. A. Aging and wound healing. World J Surg 28, 321-326, doi:10.1007/s00268-003-7397-6 (2004).
6 Gould, L. et al. Chronic wound repair and healing in older adults: current status and future research. J Am Geriatr Soc 63, 427-438, doi:10.1111/jgs.13332 (2015).
7 Emery, C. F., Kiecolt-Glaser, J. K., Glaser, R., Malarkey, W. B. & Frid, D. J. Exercise accelerates wound healing among healthy older adults: a preliminary investigation. J Gerontol A Biol Sci Med Sci 60, 1432-1436, doi:10.1093/gerona/60.11.1432 (2005).
8 Mahmoudi, S. et al. Heterogeneity in old fibroblasts is linked to variability in reprogramming and wound healing. Nature 574, 553-558 (2019).
9 Cosgrove, B. D. et al. Rejuvenation of the muscle stem cell population restores strength to injured aged muscles. Nat Med 20, 255-264, doi:10.1038/nm.3464 (2014).
10 Pennington, K. L. & DeAngelis, M. M. Epigenetic Mechanisms of the Aging Human Retina. J Exp Neurosci 9, 51-79, doi:10.4137/JEN.S25513 (2015).
11 Howard, D., Buttery, L. D., Shakesheff, K. M. & Roberts, S. J. Tissue engineering: strategies, stem cells and scaffolds. J Anat 213, 66-72, doi:10.1111/j.1469-7580.2008.00878.x (2008).
12 Franceschi, C., Garagnani, P., Parini, P., Giuliani, C. & Santoro, A. Inflammaging: a new immune-metabolic viewpoint for age-related diseases. Nat Rev Endocrinol 14, 576-590, doi:10.1038/s41574-018-0059-4 (2018).
13 Castle, S. C. Clinical relevance of age-related immune dysfunction. Clin Infect Dis 31, 578-585, doi:10.1086/313947 (2000).
14 Nikolich-Zugich, J. Ageing and life-long maintenance of T-cell subsets in the face of latent persistent infections. Nat Rev Immunol 8, 512-522, doi:10.1038/nri2318 (2008).
15 Pang, W. W. et al. Human bone marrow hematopoietic stem cells are increased in frequency and myeloid-biased with age. Proc Natl Acad Sci USA 108, 20012-20017, doi:10.1073/pnas.1116110108 (2011).
16 Badylak, S. F., Freytes, D. O. & Gilbert, T. W. Extracellular matrix as a biological scaffold material: Structure and function. Acta Biomater 5, 1-13, doi:10.1016/j.actbio.2008.09.013 (2009).
17 Brown, B. N. et al. Macrophage phenotype as a predictor of constructive remodeling following the implantation of biologically derived surgical mesh materials. Acta Biomater 8, 978-987, doi:10.1016/j.actbio.2011.11.031 (2012).
18 Mase, V. J., Jr. et al. Clinical application of an acellular biologic scaffold for surgical repair of a large, traumatic quadriceps femoris muscle defect. Orthopedics 33, 511, doi:10.3928/01477447-20100526-24 (2010).
19 Lee, K., Silva, E. A. & Mooney, D. J. Growth factor delivery-based tissue engineering: general approaches and a review of recent developments. J R Soc Interface 8, 153-170, doi:10.1098/rsif.2010.0223 (2011).
20 Teodori, L. et al. Native extracellular matrix: a new scaffolding platform for repair of damaged muscle. Front Physiol 5, 218, doi:10.3389/fphys.2014.00218 (2014).
21 Wang, Z. et al. Novel biomaterial strategies for controlled growth factor delivery for biomedical applications. NPG Asia Mater 9, e435-e435 (2017).
22 Mosala Nezhad, Z. et al. Small intestinal submucosa extracellular matrix (CorMatrix®) in cardiovascular surgery: a systematic review. Interact Cardiovasc Thorac Surg 22, 839-850, doi:10.1093/icvts/ivw020 (2016).
23 Gieseck III, R. L., Wilson, M. S. & Wynn, T. A. Type 2 immunity in tissue repair and fibrosis. Nat Rev Immunol 18, 62 (2018).
24 Heredia, J. E. et al. Type 2 innate signals stimulate fibro/adipogenic progenitors to facilitate muscle regeneration. Cell 153, 376-388, doi:10.1016/j.cell.2013.02.053 (2013).
25 Sadtler, K. et al. Developing a pro-regenerative biomaterial scaffold microenvironment requires T helper 2 cells. Science 352, 366-370, doi:10.1126/science.aad9272 (2016).
26 Janson, D. G., Saintigny, G., van Adrichem, A., Mahe, C. & El Ghalbzouri, A. Different gene expression patterns in human papillary and reticular fibroblasts. J Invest Dermatol 132, 2565-2572, doi:10.1038/jid.2012.192 (2012).
27 Korosec, A. et al. Lineage Identity and Location within the Dermis Determine the Function of Papillary and Reticular Fibroblasts in Human Skin. J Invest Dermatol 139, 342-351, doi:10.1016/j.jid.2018.07.033 (2019).
28 Haydont, V., Neiveyans, V., Fortunel, N. O. & Asselineau, D. Transcriptome profiling of human papillary and reticular fibroblasts from adult interfollicular dermis pinpoints the ‘tissue skeleton’ gene network as a component of skin chrono-ageing. Mech Ageing Dev 179, 60-77, doi:10.1016/j.mad.2019.01.003 (2019).
29 Sole-Boldo, L. et al. Single-cell transcriptomes of the human skin reveal age-related loss of fibroblast priming. Commun Biol 3, 188, doi:10.1038/s42003-020-0922-4 (2020).
30 Singhmar, P. et al. The fibroblast-derived protein PI16 controls neuropathic pain. Proc Natl Acad Sci USA 117, 5463-5471, doi:10.1073/pnas.1913444117 (2020).
31 Calcagno, D. M. et al. SiglecF(HI) Marks Late-Stage Neutrophils of the Infarcted Heart: A Single-Cell Transcriptomic Analysis of Neutrophil Diversification. J Am Heart Assoc 10, e019019, doi:10.1161/JAHA.120.019019 (2021).
32 Xie, X. et al. Single-cell transcriptome profiling reveals neutrophil heterogeneity in homeostasis and infection. Nat Immunol 21, 1119-1133, doi:10.1038/s41590-020-0736-z (2020).
33 Agier, J., Efenberger, M. & Brzezinska-Blaszczyk, E. Cathelicidin impact on inflammatory cells. Cent Eur J Immunol 40, 225-235, doi:10.5114/ceji.2015.51359 (2015).
34 Zhang, X. J. et al. Distinctive structural hallmarks and biological activities of the multiple cathelicidin antimicrobial peptides in a primitive teleost fish. J Immunol 194, 4974-4987, doi:10.4049/jimmunol.1500182 (2015).
35 Kalantari, P., Bunnell, S. C. & Stadecker, M. J. The C-type Lectin Receptor-Driven, Th17 Cell-Mediated Severe Pathology in Schistosomiasis: Not All Immune Responses to Helminth Parasites Are Th2 Dominated. Front Immunol 10, 26, doi:10.3389/fimmu.2019.00026 (2019).
36 Kalantari, P. et al. CD209a Synergizes with Dectin-2 and Mincle to Drive Severe Th17 Cell-Mediated Schistosome Egg-Induced Immunopathology. Cell Rep 22, 1288-1300, doi:10.1016/j.celrep.2018.01.001 (2018).
37 Ponichtera, H. E. et al. CD209a expression on dendritic cells is critical for the development of pathogenic Th17 cell responses in murine schistosomiasis. J Immunol 192, 4655-4665, doi:10.4049/jimmunol.1400121 (2014).
38 Wang, J. et al. A Positive Feedback Loop Between Th17 Cells and Dendritic Cells in Patients with Endplate Inflammation. Immunol Invest 48, 39-51, doi:10.1080/08820139.2018.1496097 (2019).
39 Stein-O'Brien, G. L. et al. Decomposing Cell Identity for Transfer Learning across Cellular Measurements, Platforms, Tissues, and Species. Cell Syst 8, 395-411 e398, doi:10.1016/j.cels.2019.04.004 (2019).
40 Stein-O'Brien, G. L. et al. Enter the Matrix: Factorization Uncovers Knowledge from Omics. Trends Genet 34, 790-805, doi:10.1016/j.tig.2018.07.003 (2018).
41 Zhang, W. et al. Decorin is a pivotal effector in the extracellular matrix and tumour microenvironment. Oncotarget 9, 5480-5491, doi:10.18632/oncotarget.23869 (2018).
42 Havis, E. & Duprez, D. EGR1 Transcription Factor is a Multifaceted Regulator of Matrix Production in Tendons and Other Connective Tissues. Int J Mol Sci 21, doi:10.3390/ijms21051664 (2020).
43 Rosenfeldt, H., Lee, D. J. & Grinnell, F. Increased c-fos mRNA expression by human fibroblasts contracting stressed collagen matrices. Mol Cell Biol 18, 2659-2667, doi:10.1128/MCB.18.5.2659 (1998).
44 Park, D. D. et al. Resident and elicited murine macrophages differ in expression of their glycomes and glycan-binding proteins. Cell Chem Biol 28, 567-582. e564 (2021).
54 Mor-Vaknin, N., Punturieri, A., Sitwala, K. & Markovitz, D. M. Vimentin is secreted by activated macrophages. Nat Cell Biol 5, 59-63 (2003).
46 Poon, C. C. et al. Differential microglia and macrophage profiles in human IDH-mutant and -wild type glioblastoma. Oncotarget 10, 3129 (2019).
47 Gu, Z., Chhabra, A., Alard, P., Warner, D. & Kosiewicz, M. FcγRI is required for TGFβ2-treated macrophage-induced tolerance. Immunobiology 218, 1200-1206 (2013).
48 Aibar, S. et al. SCENIC: single-cell regulatory network inference and clustering. Nat Methods 14, 1083-1086, doi:10.1038/nmeth.4463 (2017).
49 Cherry, C. et al. Intercellular signaling dynamics from a single cell atlas of the biomaterials response. bioRxiv (2020).
50 Yin, H., Price, F. & Rudnicki, M. A. Satellite cells and the muscle stem cell niche. Physiol Rev 93, 23-67, doi:10.1152/physrev.00043.2011 (2013).
51 Bouzeyen, R., Haoues, M., Barbouche, M.-R., Singh, R. & Essafi, M. FOXO3 transcription factor regulates IL-10 expression in Mycobacteria-infected macrophages, tuning their polarization and the subsequent adaptive immune response. Front Immunol 10, 2922 (2019).
52 Ciofani, M. et al. A validated regulatory network for Th17 cell specification. Cell 151, 289-303, doi:10.1016/j.cell.2012.09.016 (2012).
53 Qi, H. et al. Foxo3 Promotes the Differentiation and Function of Follicular Helper T Cells. Cell Rep 31, 107621, doi:10.1016/j.celrep.2020.107621 (2020).
54 Lin, P. Y., Jen, H. Y., Chiang, B. L., Sheu, F. & Chuang, Y. H. Interleukin-21 suppresses the differentiation and functions of T helper 2 cells. Immunology 144, 668-676, doi:10.1111/imm.12419 (2015).
55 Martinez, G. J., Nurieva, R. I., Yang, X. O. & Dong, C. Regulation and function of proinflammatory TH17 cells. Ann N Y Acad Sci 1143, 188-211, doi:10.1196/annals.1443.021 (2008).
56 Zhu, J., Cote-Sierra, J., Guo, L. & Paul, W. E. Stat5 activation plays a critical role in Th2 differentiation. Immunity 19, 739-748, doi:10.1016/s1074-7613(03)00292-9 (2003).
57 Laurence, A. et al. Interleukin-2 signaling via STATS constrains T helper 17 cell generation. Immunity 26, 371-381, doi:10.1016/j.immuni.2007.02.009 (2007).
58 Kuwahara, M. et al. Bach2-Batf interactions control Th2-type immune response by regulating the IL-4 amplification loop. Nat Commun 7, 12596, doi:10.1038/ncomms12596 (2016).
59 Lee, W., Kim, H. S., Hwang, S. S. & Lee, G. R. The transcription factor Batf3 inhibits the differentiation of regulatory T cells in the periphery. Exp Mol Med 49, e393, doi:10.1038/emm.2017.157 (2017).
60 Ho, I.-C., Lo, D. & Glimcher, L. H. c-maf promotes T helper cell type 2 (Th2) and attenuates Th1 differentiation by both interleukin 4-dependent and -independent mechanisms. J Exp Med 188, 1859-1866 (1998).
61 Kim, J. I., Ho, I.-C., Grusby, M. J. & Glimcher, L. H. The transcription factor c-Maf controls the production of interleukin-4 but not other Th2 cytokines. Immunity 10, 745-751 (1999).
62 Homma, Y., Cao, S., Shi, X. & Ma, X. The Th2 transcription factor c-Maf inhibits IL-12p35 gene expression in activated macrophages by targeting NF-kappaB nuclear translocation. J Interferon Cytokine Res 27, 799-808, doi:10.1089/jir.2007.0006 (2007).
63 Kaiko, G. E., Horvat, J. C., Beagley, K. W. & Hansbro, P. M. Immunological decision-making: how does the immune system decide to mount a helper T-cell response?Immunology 123, 326-338 (2008).
64 Chen, E. Y. et al. Enrichr: interactive and collaborative HTML5 gene list enrichment analysis tool. BMC Bioinformatics 14, 128, doi:10.1186/1471-2105-14-128 (2013).
65 Kuleshov, M. V. et al. Enrichr: a comprehensive gene set enrichment analysis web server 2016 update. Nucleic Acids Res 44, W90-97, doi:10.1093/nar/gkw377 (2016).
66 Schmidt-Weber, C. B., Akdis, M. & Akdis, C. A. TH17 cells in the big picture of immunology. J Allergy Clin Immunol 120, 247-254, doi:10.1016/j.jaci.2007.06.039 (2007).
67 Ohnmacht, C. et al. The microbiota regulates type 2 immunity through RORγt+ T cells. Science 349, 989-993 (2015).
68 Liu, T., Zhang, L., Joo, D. & Sun, S.-C. NF-κB signaling in inflammation. Signal Transduct Target Ther 2, 1-9 (2017).
69 Xu, S. & Cao, X. Interleukin-17 and its expanding biological functions. Cell Mol Immunol 7, 164-174, doi:10.1038/cmi.2010.21 (2010).
70 Sutton, C., Brereton, C., Keogh, B., Mills, K. H. & Lavelle, E. C. A crucial role for interleukin (IL)-1 in the induction of IL-17-producing T cells that mediate autoimmune encephalomyelitis. J Exp Med 203, 1685-1691 (2006).
71 Bunte, K. & Beikler, T. Th17 Cells and the IL-23/IL-17 Axis in the Pathogenesis of Periodontitis and Immune-Mediated Inflammatory Diseases. Int J Mol Sci 20, 3394, doi:10.3390/ijms20143394 (2019).
72 Fabre, T. et al. Type 3 cytokines IL-17A and IL-22 drive TGF-beta-dependent liver fibrosis. Sci Immunol 3, doi:10.1126/sciimmunol.aar7754 (2018).
73 Tesmer, L. A., Lundy, S. K., Sarkar, S. & Fox, D. A. Th17 cells in human disease. Immunol Rev 223, 87-113, doi:10.1111/j.1600-065X.2008.00628.x (2008).
74 Malecova, B. et al. Dynamics of cellular states of fibro-adipogenic progenitors during myogenesis and muscular dystrophy. Nat Commun 9, 3670, doi:10.1038/s41467-018-06068-6 (2018).
75 Yang, X. O. et al. T helper 17 lineage differentiation is programmed by orphan nuclear receptors RORα and RORγ. Immunity 28, 29-39 (2008).
76 Folker, E. S. & Baylies, M. K. Nuclear positioning in muscle development and disease. Front Physiol 4, 363, doi:10.3389/fphys.2013.00363 (2013).
77 Cadot, B., Gache, V. & Gomes, E. R. Moving and positioning the nucleus in skeletal muscle-one step at a time. Nucleus 6, 373-381 (2015).
78 Prasad, S., Sung, B. & Aggarwal, B. B. Age-associated chronic diseases require age-old medicine: role of chronic inflammation. Prev Med 54 Suppl, S29-37, doi:10.1016/j.ypmed.2011.11.011 (2012).
79 Pal, S. & Tyler, J. K. Epigenetics and aging. Sci Adv 2, e1600584, doi:10.1126/sciadv.1600584 (2016).
80 Wagoner, Z. W. & Zhao, W. Therapeutic implications of transplanted-cell death. Nat Biomed Eng 5, 379-384, doi:10.1038/s41551-021-00729-6 (2021).
81 Liu, L. et al. Evaluation of the biocompatibility and mechanical properties of xenogeneic (porcine) extracellular matrix (ECM) scaffold for pelvic reconstruction. Int Urogynecol J 22, 221-227, doi:10.1007/s00192-010-1288-9 (2011).
82 Chow, D. W. & Westermeyer, H. D. Retrospective evaluation of corneal reconstruction using AC ell Vet™ alone in dogs and cats: 82 cases. Vet Ophthalmol 19, 357-366 (2016).
83 Sadtler, K. et al. Developing a pro-regenerative biomaterial scaffold microenvironment requires T helper 2 cells. Science 352, 366-370 (2016).
84 Satija, R., Farrell, J. A., Gennert, D., Schier, A. F. & Regev, A. Spatial reconstruction of single-cell gene expression data. Nat Biotechnol 33, 495-502, doi:10.1038/nbt.3192 (2015).
85 Tirosh, I. et al. Dissecting the multicellular ecosystem of metastatic melanoma by single-cell RNA-seq. Science 352, 189-196, doi:10.1126/science.aad0501 (2016).
86 Aibar, S. et al. SCENIC: single-cell regulatory network inference and clustering. Nat Methods 14, 1083-1086, doi:10.1038/nmeth.4463 (2017).
87 Stein-O'Brien, G. L. et al. Decomposing Cell Identity for Transfer Learning across Cellular Measurements, Platforms, Tissues, and Species. Cell Syst 8, 395-411 e398, doi:10.1016/j.cels.2019.04.004 (2019).
88 Faust, H. J., et al. IL-17 and immunologically induced senescence regulate response to injury in osteoarthritis. J Clin Invest 130, 5493-5507 (2020).
89 Grunewald, M., et al. Counteracting age-related VEGF signaling insufficiency promotes healthy aging and extends life span. Science 373(2021).

What is claimed is:

1. A method for promoting wound healing, tissue repair, or tissue regeneration, in a subject in need thereof, the method comprising administering a therapeutically effective amount of at least one pharmaceutical composition comprising at least one IL-17 antagonist and at least one regenerative therapy to the subject.

2. The method of claim 1, wherein the at least one IL-17 antagonist and regenerative therapy are administered simultaneously to the subject.

3. The method of claim 1, wherein the at least one IL-17 antagonist and regenerative therapy are administered sequentially to the subject.

4. The method of claim 1, wherein the IL-17 antagonist, regenerative therapy, or the IL-antagonist and regenerative therapy are administered systemically to the subject.

5. The method of claim 1, wherein the at least one IL-17 antagonist, regenerative therapy, or the IL-antagonist and regenerative therapy are administered locally to the site of the wound or area of tissue repair or regeneration in the subject.

6. The method of claim 1, wherein the subject has one or more inhibitory factors that inhibit or prevent regeneration.

7. The method of claim 6, wherein the inhibitory factors may be age, infection, autoimmune disease or any combination thereof.

8. The method of claim 1, wherein the IL-17 antagonist is an IL-17 antibody or an antigen-binding portion thereof.

9. The method of claim 8, wherein the IL-17 antibody, or antigen-binding portion thereof, is a monoclonal antibody, a chimeric antibody, a bi-specific antibody, a human antibody, or antigen-binding portion thereof.

10. The method of claim 9, wherein the IL-17 antibody, or antigen-binding portion thereof, is a human antibody.

11. The method of claim 10, wherein the human antibody, or antigen-binding portion thereof, can specifically bind to human IL-17A, human 1L-17F and/or human IL-17A/F.

12. The method of claim 1, wherein the at least one regenerative therapy is stem cells, platelet-rich plasma, extracellular matrix (ECM), prolotherapy, lipogems, or any combinations thereof.

13. The method of claim 1, wherein the method further comprises a single pharmaceutical composition containing at least one IL-17 antagonist and at least one regenerative therapy.

14. The method of claim 13, wherein the pharmaceutical composition is a delayed-release composition.

15. The method of claim 1, wherein the method further comprises a first pharmaceutical composition containing at least one IL-17 antagonist and a second pharmaceutical composition containing at least one regenerative therapy.

16. A kit for use in promoting wound healing, tissue repair, or tissue regeneration, in a subject in need thereof, the kit comprising at least one pharmaceutical composition comprising at least one IL-17 antagonist and at least one regenerative therapy.

17. The kit of claim 16, wherein the IL-17 antagonist is an IL-17 antibody or an antigen-binding portion thereof.

18. The kit of claim 17, wherein the IL-17 antibody, or antigen-binding portion thereof, is a monoclonal antibody, a chimeric antibody, a bi-specific antibody, a human antibody, or antigen-binding portion thereof.

19. The kit of claim 18, wherein the IL-17 antibody, or antigen-binding portion thereof, is a human antibody.

20. The kit of claim 16, wherein the at least one regenerative therapy is stem cells, platelet-rich plasma, extracellular matrix (ECM), prolotherapy, lipogems, or any combinations thereof.

21. The kit of claim 16, wherein the kit comprises a single pharmaceutical composition containing at least one IL-17 antagonist and at least one regenerative therapy.

22. The kit of claim 21, wherein the pharmaceutical composition is a delayed-release composition.

23. The kit of claim 16, wherein the kit comprises a first pharmaceutical composition containing at least one IL-17 antagonist and a second pharmaceutical composition containing at least one regenerative therapy.
